(19) United States
(12) Patent Application Publication
(10) Pub. No.: US 2026/0265060 A1
(43) Pub. Date: Sep. 10, 2026

(54) FULLERENE DERIVATIVE, SYNTHESIS REACTION OF FULLERENE DERIVATIVE, PRODUCTION METHOD OF FULLERENE DERIVATIVE, VAPOR-DEPOSITED PRODUCT, AND PRODUCTION METHOD OF VAPOR-DEPOSITED PRODUCT

(71) Applicant: Mitsubishi Corporation, Tokyo (JP)

(72) Inventors: Yutaka MATSUO, Aichi (JP); Haosheng LIN, Aichi (JP); Naoyuki IMAI, Tokyo (JP); Takeshi IGARASHI, Tokyo (JP)

(21) Appl. No.: 19/165,186

(22) PCT Filed: Mar. 19, 2024
(86) PCT No.: PCT/JP2024/010724

§ 371 Date: Sep. 15, 2025

(30) Foreign Application Priority Data
Mar. 24, 2023 (JP) 2023-048815
May 12, 2023 (JP) 2023-205171

(51) Int. Cl. C01B 32/05; C07C 49/617; C07C 69/616; C23C 14/12; C23C 14/24

(52) U.S. Cl. CPC C01B 32/05; C07C 49/617; C07C 69/616; C23C 14/12; C23C 14/24; C01P 2002/02; C07C 2603/54; C07C 2604/00

(57) ABSTRACT

A fullerene derivative is represented by a formula (1):

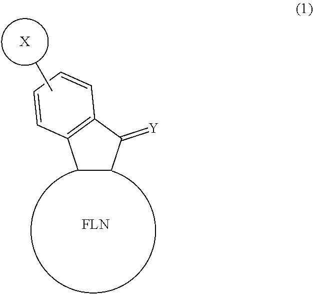

in the formula (1), FLN is a fullerene framework, X is a halogen group or an organic group, and Y is O or S.

### TECHNICAL FIELD

The present invention relates to fullerene derivatives, synthesis reactions of fullerene derivatives, production methods of fullerene derivatives, vapor-deposited products, and production methods of vapor-deposited products.

### BACKGROUND ART

In development of devices, such as photoelectric conversion elements or image sensors, a technology using a fullerene or fullerene derivative has attracted attention. The fullerene or fullerene derivative has high light absorption and desired electrical characteristics, and can be vacuum-deposited. For a fullerene or fullerene derivative, a fullerene derivative having a structure in which indanone is condensed with a fullerene is disclosed in Patent Document 1.

### RELATED ART DOCUMENTS

### Patent Documents

Patent Document 1: International Publication No. WO 2021/149801

### DISCLOSURE OF THE INVENTION

### Problems to be Solved by the Invention

There are some fullerene derivatives that can be vapor-deposited, but vapor-deposited films including these fullerene derivatives do not have an amorphous structure. Therefore, it is difficult to mount such vapor-deposited films on various devices.

An object of the present disclosure is to provide a fullerene derivative that can be formed into a vapor-deposited film having an amorphous structure.

### Means for Solving the Problems

A fullerene derivative of the present disclosure is represented by a formula (1):

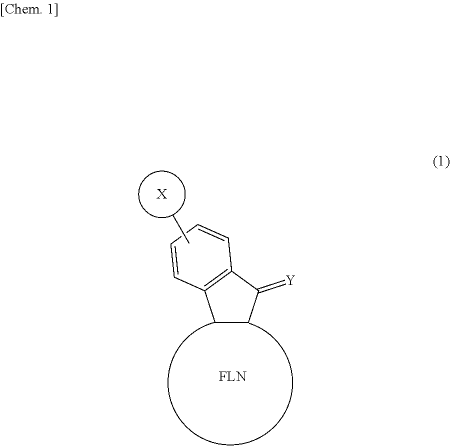

(in the formula, FLN is a fullerene framework, X is a halogen group or an organic group, and Y is O or S).

### Effects of the Invention

According to one aspect of the present invention, a fullerene derivative that can be formed into a vapor-deposited film having an amorphous structure can be provided.

### BRIEF DESCRIPTION OF THE DRAWINGS

FIG. 1 is a diagram illustrating a structural formula of tert-butyl indanofullerene ketone (tBu-FIDO).

FIG. 2 is a diagram illustrating a synthetic route of tBu-FIDO.

FIG. 3 is a diagram illustrating the synthetic route of tBu-FIDO (synthetic route from a dimer to ketone).

FIG. 4 is a 1H NMR chart of a 2-ethoxy-5-tert-butylphenyl hydrofullerene (intermediate product).

FIG. 5 is an enlarged view of a part of the chart of FIG. 4.

FIG. 6 is a 13C NMR chart of a 2-ethoxy-5-tert-butylphenyl hydrofullerene (intermediate product).

FIG. 7 is an enlarged view of a part of the chart of FIG. 6.

FIG. 8 is a 1H NMR chart of tBu-FIDO.

FIG. 9 is an enlarged view of a part of the chart of FIG. 8.

FIG. 10 is a 13C NMR chart of tBu-FIDO.

FIG. 11 is a graph depicting reduction potential of a fullerene (C₆₀) and fullerene derivatives.

FIG. 12 is a table illustrating reduction potential and electron affinity of a fullerene and fullerene derivatives.

FIG. 13 is a diagram illustrating a structural formula of a fullerene (C₆₀).

FIG. 14 is a diagram illustrating a structural formula of indanofullerene ketone (H-FIDO).

FIG. 15 is a diagram illustrating a structural formula of methoxy indanofullerene ketone (MeO-FIDO).

FIG. 16 is a diagram illustrating a structural formula of fluoroindanofullerene ketone (F-FIDO).

FIG. 17 is a diagram illustrating a structural formula of methyl indanofullerene ketone (Me-FIDO).

FIG. 18 is a graph illustrating X-ray diffraction patterns of C₆₀ immediately after vapor deposition of C₆₀ before annealing, and after annealing.

FIG. 19 is a graph illustrating X-ray diffraction patterns of H-FIDO immediately after vapor deposition of H-FIDO before annealing, and after annealing.

FIG. 20 is a graph illustrating X-ray diffraction patterns of Me-FIDO immediately after vapor deposition of Me-FIDO before annealing, and after annealing.

FIG. 21 is a graph illustrating X-ray diffraction patterns of tBu-FIDO immediately after vapor deposition of tBu-FIDO before annealing, and after annealing.

FIG. 22 illustrates transmission electron microscope (TEM) images (left and upper right) and an X-ray diffraction image (bottom right) of tBu-FIDO.

FIG. 23 illustrates TEM images of H-FIDO.

FIG. 24 illustrates TEM images (left and upper right) and an X-ray diffraction image (bottom right) of tBu-FIDO after annealing.

FIG. 25 illustrates TEM images (left and upper right) and an X-ray diffraction image (bottom right) of H-FIDO after annealing.

FIG. 26 is a graph illustrating a thermogravimetric analysis (TGA) chart of C₆₀.

FIG. 27 is a graph illustrating a TGA chart of [6,6]-phenyl-C₆₁-methyl butyrate (PC₆₁BM).

FIG. 28 is a graph illustrating a TGA chart of H-FIDO.

FIG. 29 is a graph illustrating a TGA chart of Me-FIDO.

FIG. 30 is a graph illustrating a TGA chart of tBu-FIDO.

FIG. 31 is a table illustrating results of TGA of fullerene derivatives.

FIG. 32 is a table illustrating electric current at the time of sublimation of fullerene derivatives.

FIG. 33 is a graph illustrating X-ray diffraction charts of a fullerene and tBu-FIDO.

FIG. 34 is a graph illustrating X-ray diffraction charts of a tBu-FIDO film one month after vapor deposition, one month after annealing performed after vapor deposition, and one month after solvent vapor exposure performed after vapor deposition.

FIG. 35 is a graph illustrating X-ray diffraction charts of a fullerene film one day after vapor deposition, one day after annealing performed after vapor deposition, one day after solvent vapor exposure performed after vapor deposition, one month after vapor deposition, one month after annealing performed after vapor deposition, and one month after solvent vapor exposure performed after vapor deposition.

FIG. 36 is a graph illustrating a relationship between a wavelength and absorbance in ultraviolet-visible (UV-vis) spectroscopy of a fullerene and tBu-FIDO.

FIG. 37 is a table illustrating results of vacuum thermogravimetry (vacuum TG) of fullerene derivatives.

FIG. 38 is a diagram illustrating a structural formula of tert-butyl indanofullerene thioketone (tBu-FIDS).

FIG. 39 is a diagram illustrating a synthetic route of tBu-FIDS (reaction passage from tBu-FIDO to tBu-FIDS).

FIG. 40 is a 1H NMR chart of tBu-FIDS.

FIG. 41 is a 13C NMR chart of tBu-FIDS.

FIG. 42 is a 13C NMR chart of tBu-FIDS.

FIG. 43 is a graph illustrating a differential thermal analysis (DTA) chart of tBu-FIDS.

FIG. 44 is a table illustrating results of thermogravimetry (TG) of tBu-FIDS, C₆₀, and tBu-FIDO.

FIG. 45 is a graph illustrating reduction potential of Cao, tBu-FIDS, and tBu-FIDO.

FIG. 46 is a table illustrating reduction potential and electron affinity of Coo, tBu-FIDO, and tBu-FIDS.

FIG. 47 is a graph illustrating simulation values and measured values of electron affinity of C₆₀, tBu-FIDO, and tBu-FIDS.

### MODE OF CARRYING OUT THE INVENTION

Embodiments of the present invention will be described with reference to drawings hereinafter.

### <Fullerene Derivative>

The fullerene derivative of the present disclosure is represented by the above formula (1).

FLN in the above formula (1) is a fullerene framework.

Examples of the fullerene framework include a C₆₀ framework, a C₇₀ framework, a C₇₆ framework, C₇₈ framework, higher-order fullerene frameworks, and the like. Among the above frameworks, the Coo framework is preferable. Since Coo serving as a starting material can be easily industrially obtained with a high purity compared to other fullerenes, a purity of a fullerene derivative derived from C₆₀ can be increased.

As presented in the above formula (1), the fullerene derivative of the present disclosure has an indanofullerene structure in which an indanone (C₉H₈O) is condensed with a fullerene framework.

Moreover, X in the above formula (1) is a halogen group or an organic group.

Examples of the halogen group include a chlorine atom, a bromine atom, an iodine atom, and the like. Among the above halogen groups, the chlorine atom is preferable.

The organic group is, for example, an alkyl group, a heteroalkyl group, an alkenyl group, a heteroalkenyl group, an alkynyl group, a heteroalkynyl group, a cycloalkyl group, a heterocycloalkyl group, an aryl group, a heteroaryl group, an alkoxy group, a carbonyl group, an imino group, or the like, or any combination of two or more of the foregoing. If the organic group is not cyclic, the organic group may be a straight chain group or a branched chain group. Moreover, the organic group may have a substituent.

The carbon number of the organic group is preferably 1 or greater and 10 or less, more preferably 2 or greater and 8 or less, and yet more preferably 3 or greater and 6 or less. Among the above-listed organic groups, the organic group is preferably an alkyl group or an alkoxy group, more preferably an alkyl group having a carbon number of 1 or greater and 10 or less, and yet more preferably a branched alkyl group having a carbon number of 3 or greater and 6 or less. Examples of the branched alkyl group having the carbon number of 3 or greater and 6 or less include a tert-butyl group.

Y in the formula (1) is oxygen (O) or sulfur(S) . In the case where Y is 0, the fullerene derivative represented by the above formula (1) has an indanofullerene ketone structure. The indanofullerene ketone may be also referred to as fullerene indanone ketone. In the case where Y is S, the fullerene derivative represented by the above formula (1) has an indanofullerene thioketone structure.

The fullerene derivative represented by the above formula (1) is preferably a fullerene derivative represented by the following formula (2). The fullerene derivative represented by the following formula (2) has an indanofullerene ketone structure.

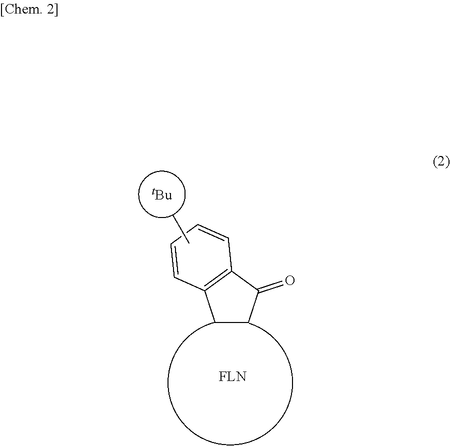

In the formula (2), tBu is a tert-butyl group. The bonding position of tBu is not particularly limited. For example, tBu may be bonded to any of the 1- to 4-positions of the indanone framework constituting the fluorene derivative represented by the above formula (2). The bonding position of tBu is controlled by constituent substances for use or synthesis conditions.

The fullerene derivative represented by the above formula (2) is preferably a fullerene derivative represented by the following formula (3).

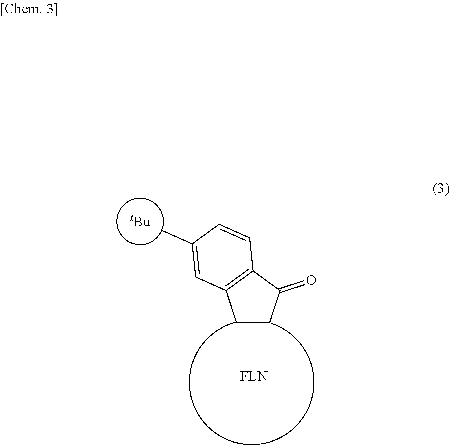

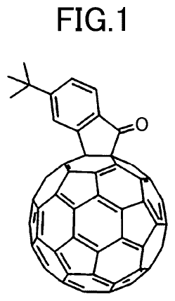

In the fullerene derivative represented by the above formula (3), tBu is bonded to the 2-position (B-position) of the indanone framework constituting the fullerene derivative represented by the above formula (2). The fullerene derivative represented by the above formula (2) corresponds to tert-butyl indanofullerene ketone depicted in FIG. 1. In the present specification, tert-butyl indanofullerene ketone may be abbreviated as tBu-FIDO.

Moreover, the fullerene derivative represented by the above formula (1) is preferably a fullerene derivative represented by the following formula (4). The fullerene derivative represented by the following formula (4) has an indanofullerene thioketone structure.

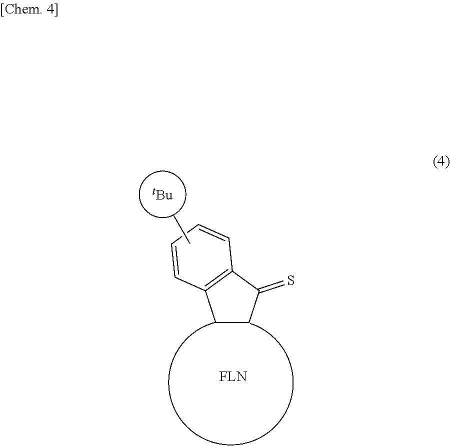

In the formula (4), tBu is a tert-butyl group. The bonding position of tBu is not particularly limited. For example, tBu may be bonded to any of the 1- to 4-positions of the indanone framework constituting the fullerene derivative represented by the above formula (4). The bonding position of tBu is controlled by constituent substances for use or synthesis conditions. The fullerene derivative represented by the above formula (4) is preferably a fullerene derivative represented by the following formula (5).

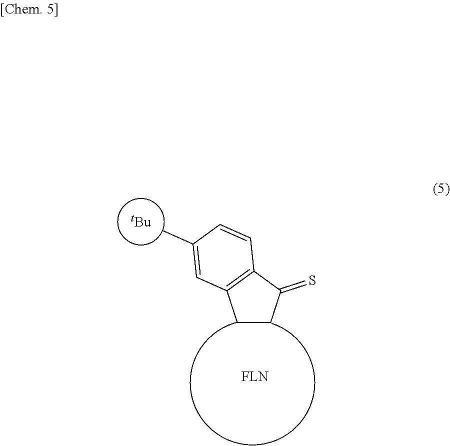

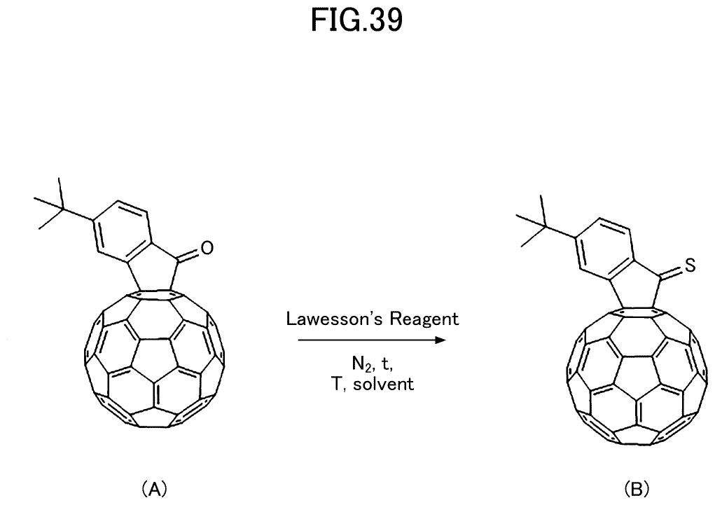

In the fullerene derivative represented by the above formula (5), tBu is bonded to the 2-position (B-position) of the indanone framework constituting the fullerene derivative represented by the above formula (4). The fullerene derivative represented by the above formula (2) corresponds to tert-butyl indanofullerene thioketone illustrated in FIG. 38. In the present specification, tert-butyl indanofullerene thioketone may be abbreviated as tBu-FIDS.

Since the fullerene derivative of the present disclosure is a fullerene derivative represented by any of the above formulae (1) to (5) as described above, a vapor deposition including the fullerene derivative can create an amorphous structure. Thus, application of the fullerene derivative of the present disclosure in various devices can be expected.

Since the fullerene derivative of the present disclosure is a fullerene derivative represented by the above formula (2) as described above, a vapor-deposited film including the fullerene derivative can create an amorphous structure, and the amorphous structure of the vapor-deposited film can be maintained even after annealing or exposure to a solvent vapor. The annealing is performed by applying heat to the vapor-deposited product to remove the residual stress of the vapor-deposited product, thereby inhibiting deformation of the vapor-deposited product. The annealing may be performed in a solvent vapor.

Specifically, the fullerene derivative represented by the above formula (2) is unlikely to become crystalline at the time of annealing or exposure to a solvent vapor after vapor deposition, thereby avoiding a problem of a device, which may be caused by crystallization of the fullerene derivative when the device is fabricated.

Since the fullerene derivative of the present disclosure is a fullerene derivative represented by the above formula (3) as described above, the amorphous structure of the vapor-deposited film including the fullerene derivative can be maintained for a long period. Moreover, owing to the fullerene derivative of the present disclosure, the amorphous structure of the vapor-deposited film including the fullerene derivative can be maintained for a long period even after annealing or exposure to a solvent vapor.

Since the fullerene framework of the fullerene derivative of the present disclosure represented by any of the above formulae (1) to (5) is at least one selected from the group consisting of a C₆₀ framework, a C₇₀ framework, a C₇₆ framework, and a C₇₈ framework, a sublimation temperature of the vapor-deposited product including the fullerene framework can be made lower than a sublimation temperature of the fullerene, while maintaining the amorphous structure of the vapor-deposited film including the fullerene derivative for a long period. Further, when the fullerene derivatives represented by the above formulae (2) and (3) are used, the sublimation temperature of the vapor-deposited product including the fullerene framework can be made lower than a sublimation temperature of the fullerene, while maintaining the amorphous structure for a long period even after annealing or exposure to a solvent vapor.

### <Synthesis Reaction and Production Method of Fullerene Derivative>

The fullerene derivative of the present disclosure can be produced through a below-described synthesis reaction of a fullerene derivative.

The synthesis reaction of the fullerene derivative is, for example, a synthesis reaction of a fullerene derivative having an indanofullerene ketone structure (the case where Y is oxygen) among fullerene derivatives represented by the above formula (1). The above reaction is a synthesis reaction of a fullerene derivative represented by the following formula (6), in which an aryl fullerene dimer is oxidized in the presence of hydrohalic acid.

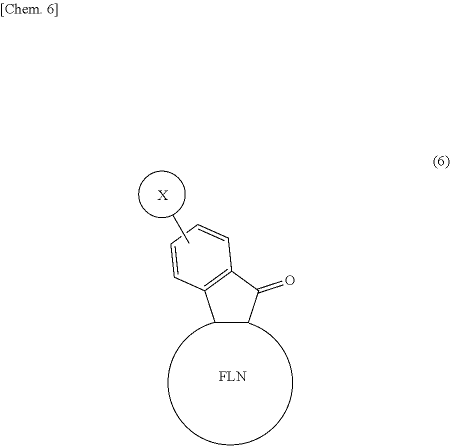

In the formula (6), FLN is a fullerene framework, and X is a hydrogen, a halogen group, or an organic group. In the case where X is a hydrogen, an aryl group to which X is bonded in the fullerene derivative represented by the above formula (6) does not have a substituent. Specifically, an aryl group to which X is bonded in the fullerene derivative represented by the above formula (6) optionally has a substituent.

The aryl fullerene dimer is a fullerene derivative precursor synthesized by oxidizing an aryl(hydro)fullerene, which is synthesized by a reaction between a Grignard reagent and a fullerene, in the presence of a base.

The hydrohalic acid is an aqueous solution of a hydrogen halide. Examples of the hydrogen halide include hydrogen chloride, hydrogen bromide, and the like. Among hydrohalic acids, hydrobromic acid, which is an aqueous solution of hydrogen bromide, is preferable in view of reproducibility of a resultant fullerene derivative and improvement in yield.

Moreover, a concentration of the hydrohalic acid as an aqueous solution is preferably 35% to 60%, more preferably 40% to 55%, and yet more preferably 45% to 50%. When the hydrohalic acid has a concentration of 35% to 60%, the hydrohalic acid is easily handled, and reproducibility and yield of the resultant fullerene derivative can be further improved. In particular, when 45% to 50% hydrobromic acid is used as the hydrohalic acid, reproducibility and yield of the resultant fullerene derivative can be further improved.

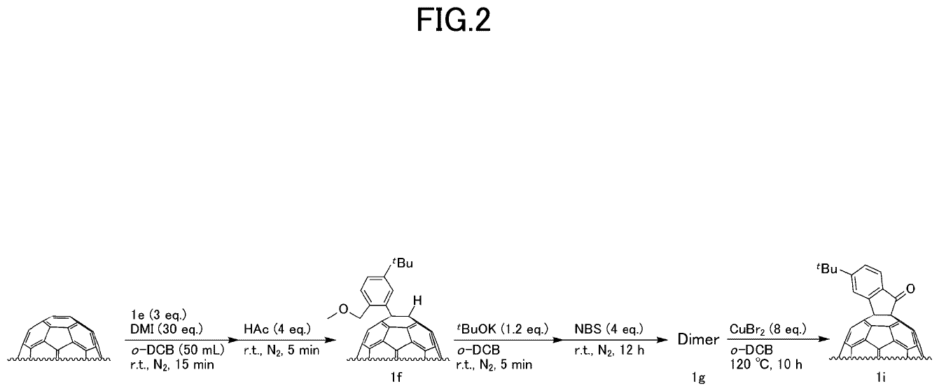

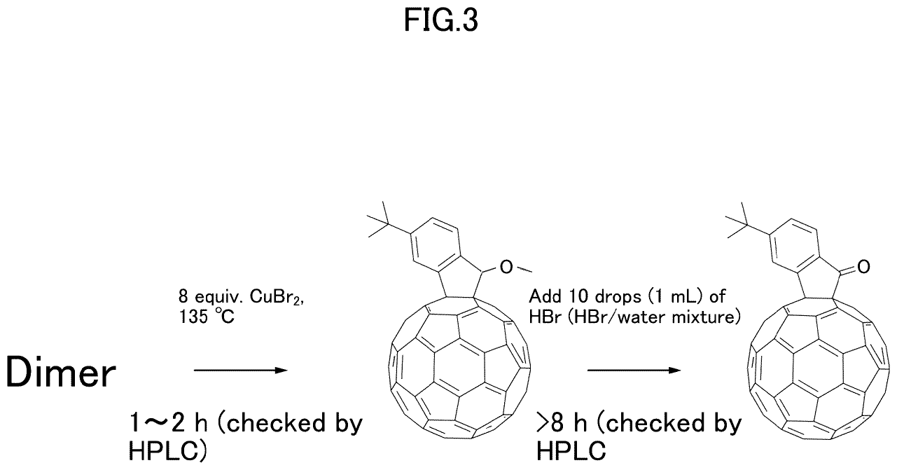

The synthesis reaction of the fullerene derivative represented by the above formula (6) will be specifically described. FIGS. 2 and 3 are diagrams illustrating a synthetic route of tert-butyl indanofullerene ketone (tBu-FIDO) that is one example of the fullerene derivative.

As illustrated in FIG. 2, first, 2-ethoxy-5-tert-butylphenyl hydrofullerene (referred to as 1f hereinafter) is synthesized. In the synthesis of 1f, C₆ (500 mg, 0.7 mmol) was dissolved in ortho-dichlorobenzene (o-DCB) (50.0 mL) including 1,3-dimethyl-2-imidazolidinone (DMI) (2.3 mL, 21 mmol), and degassing and replenishment of nitrogen (N₂) were performed three times. Subsequently, a Grignard reagent (2.0 mL, 2.1 mmol) was added in an N₂ atmosphere at room temperature.

The Grignard reagent was synthesized in the following manner. First, an anhydrous tetrahydrofuran (THF) (10.0 mL) solution of 2-bromo-4-(tert-butyl)-1-(methoxymethyl)benzene (2.1 mL, 10.0 mmol) was slowly dripped to a polished magnesium (Mg) powder (360.0 mg, 15 mmol) serving as an initiator in a small amount of iodine (I₂). Thereafter, the resultant mixture was vigorously stirred for 1 hour to thereby obtain a Grignard solution. The Grignard solution was transferred by Schlenk technique, and stocked in a Schlenk flask. The concentration of the Grignard reagent was determined to be 1.04 mol/L by anhydrous titration. The anhydrous titration was performed in an N₂ atmosphere using a small amount of 1, 10-phenanthroline as an indicator and methanol as a titrant.

Next, the Grignard reagent was added, and the resultant mixture was stirred for 15 minutes at room temperature. Thereafter, to the resultant dark green reaction mixture, CH₃COOH (160 μL, 2.8 mmol) was added to terminate the reaction. The dark green reaction mixture immediately turned brown, and the solvent was evaporated in a vacuum to concentrate the reaction mixture. Next, silica gel chromatography using carbon disulphide (CS₂) as an eluent was used to obtain 2-ethoxy-5-tert-butylphenylhydrofullerene (1f) (565.8 mg, 90%) as a brownish powder.

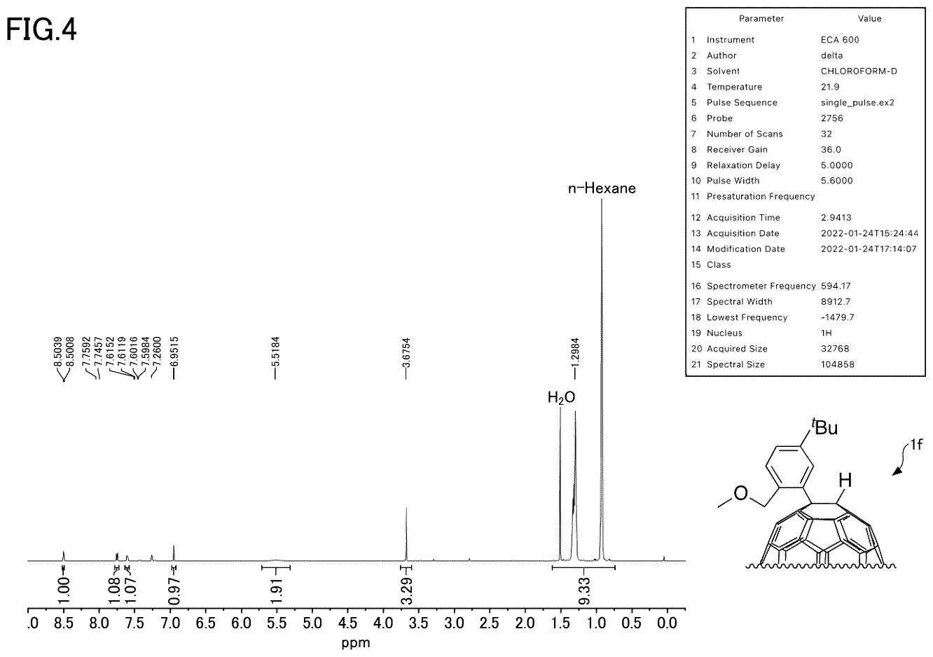

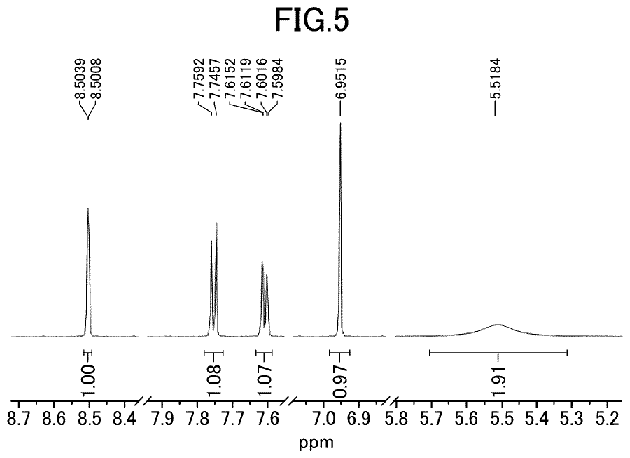

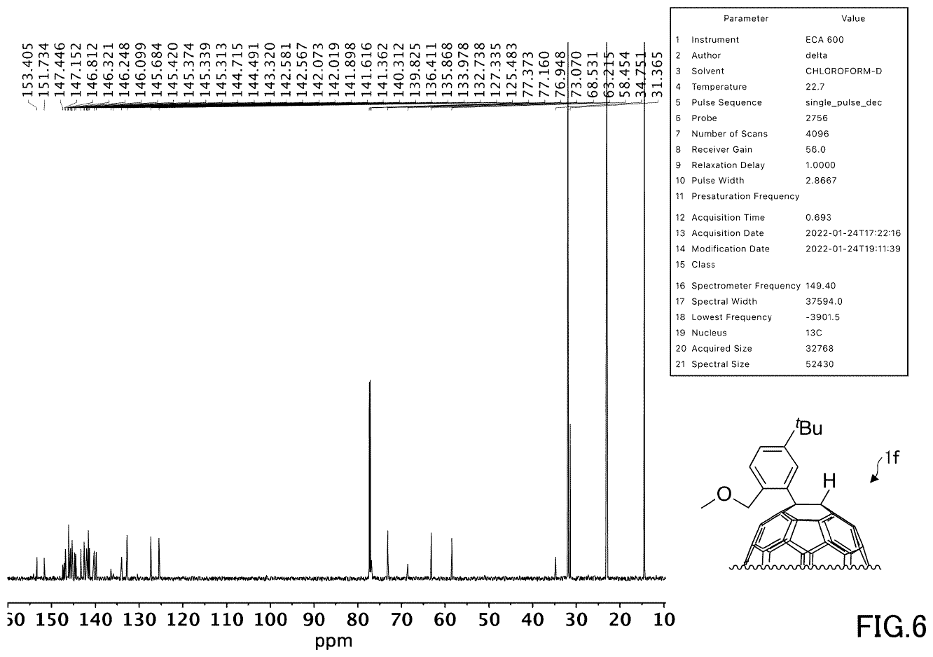

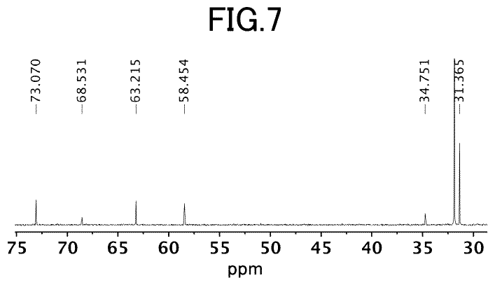

If was determined from the 1H NMR chart (see FIGS. 4 and 5) and the 13C NMR chart (see FIGS. 6 and 7). The analysis results of NMR are presented below.

1H NMR (600 MHz, CS₂/CDCl₃) δ 8.50 (d, J=2.0 Hz, 1H), 7.75 (d, J=8.1 Hz, 1H), 7.61 (dd, J=8.1 Hz, 2.0 Hz, 1H), 6.95 (s, 1H), 4.49 (s, 1H), 5.52 (s, 1H), 3.68 (s, 3H), 1.30 (s, 9H).

13C{¹H} NMR (125 MHz, CS₂/CDCl₃, all 1C unless otherwise specified) 8.50, 8.50.

Next, as illustrated in FIG. 2, an aryl fullerene dimer (may be referred to as Dimer (1g) hereinafter) is synthesized. First, a t-BuOK (67.0 μL, 0.067 mmol, 1M) THE solution was added to a 1f (50.0 mg, 0.056 mmol) solution in a 3.0 mL anhydrous 1f (50.0 mg, 0.056 mmol) solution at room temperature. After stirring the mixture solution for 15 minutes in an N₂ atmosphere, N-bromosuccinimide (NBS) (39.9 mg, 0.224 mmol) was added. The resultant reaction mixture was vigorously stirred in an N₂ atmosphere at room temperature for 12 hours. Next, the obtained brownish suspension was rapidly cooled with 0.5 mL of H₂O, and an excessive amount of MeOH was added to precipitate a crude product. Finally, Dimer (1g) was collected as a residue by filtration.

Next, tert-butyl indanofullerene ketone (tBu-FIDO) (1i) is synthesized. First, Dimer (1g) (200.0 mg, 0.112 mmol) was dissolved in o-DCB (20.0 mL), and CuBr₂ (200.0 mg, 0.896 mmol) was added as an oxidizing agent to the resultant solution. After vigorously stirring the solution at 135° C. for 1 hour, 500 μL of hydrobromic acid including hydrogen bromide at a concentration of 47% was added as hydrohalic acid. Then, the resultant mixture was vigorously stirred at 135° C. for 8 hours. Finally, the solvent of the resultant reaction mixture was evaporated in a vacuum to concentrate the reaction mixture, followed by obtaining tBu-FIDO) (1i) (105.2 mg, 53%) as a brownish powder by silica gel column chromatography (CS₂).

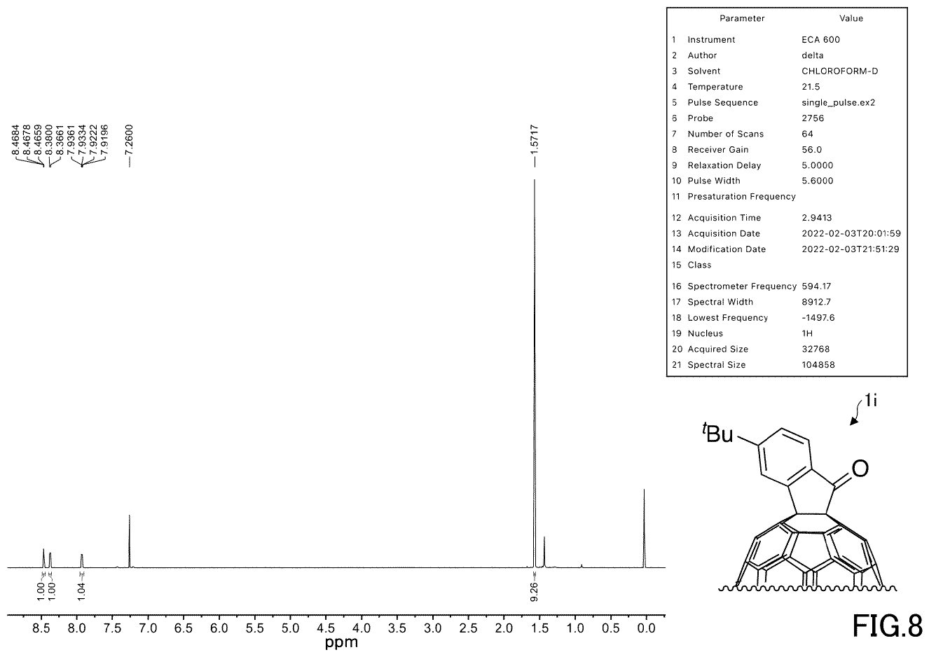

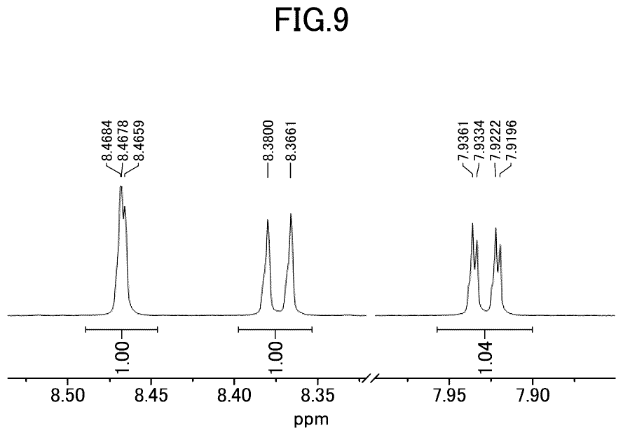

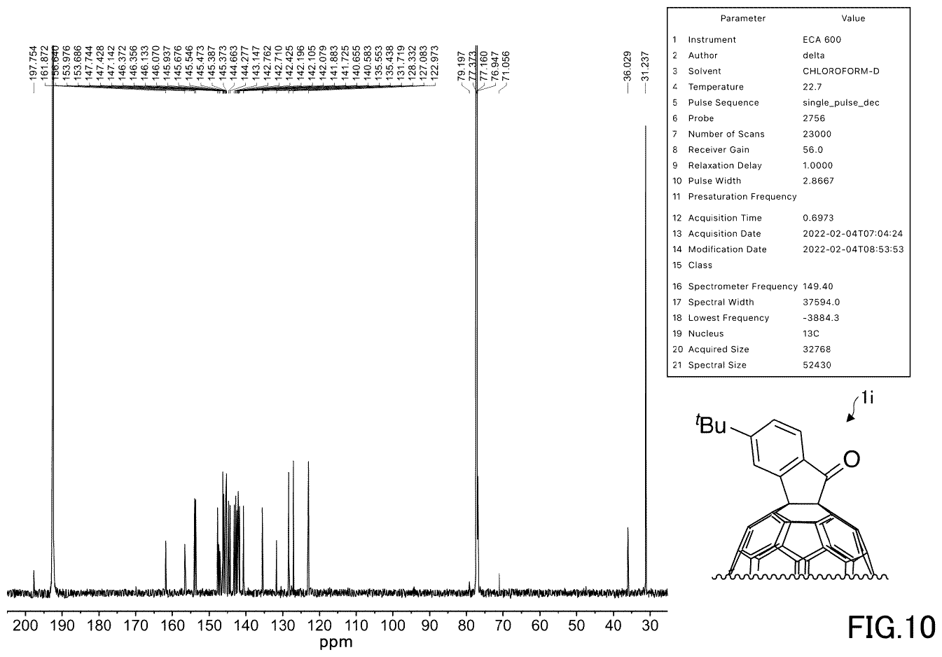

1i was determined from the 1H NMR chart (see FIGS. 8 and 9) and the 3C NMR chart (see FIG. 10). The analysis results of NMR will be presented below.

¹H NMR (600 MHz, CS₂/CDCl₃) δ8.47 (d, J=1.5 Hz, 1H), 8.37 (d, J=8.3 Hz, 1H), 7.93 (dd, J=8.3 Hz, 1.5 Hz, 1H), 1.57 (s, 9H).

13C{¹}NMR (125 MHz, CS₂/CDCl₃, all 2C unless otherwise specified) 146.4, 146.4, 146.1, 146.1, 145.9, 145.7, 145.5, 145.5, 145.4, 145.4, 144.7, 144.3, 143.1, 142.8, 142.7, 142.4, 142.2, 142.1, 142.1, 141.9, 141.7, 140.7, 140.6, 135.6, 135.4, 131.7 (1C), 128.3 (1C), 127.1 (1C), 123.0 (1C), 79.2 (1C), 71.1 (1C), 36.0 (1C), 31.2 (3C).

According to the synthesis reaction of the fullerene derivative represented by the above formula (6) of the present disclosure, as described above, the aryl fullerene dimer is oxidized in the presence of the hydrohalic acid so that the fullerene derivative represented by the above formula (6), such as tert-butyl indanofullerene ketone, can be synthesized at a high yield.

Moreover, according to the synthesis reaction of the fullerene derivative represented by the above formula (6) of the present disclosure, as described above, hydrobromic acid is used as hydrohalic acid so that the fullerene derivative represented by the above formula (6), such as tert-butyl indanofullerene ketone, can be easily synthesized at a high yield with high producibility.

Further, in the production method of the fullerene derivative of the present disclosure, the fullerene derivative is produced using the synthesis reaction of the fullerene derivative represented by the above formula (6) so that the obtained fullerene derivative can be formed into a vacuum-deposition film having an amorphous structure, and such vacuum-deposition film can maintain the amorphous structure after annealing or exposure to a solvent vapor.

The synthesis reaction of the fullerene derivative of the present disclosure is not limited to production of the fullerene derivative represented by the above formula (6), such as tert-butyl indanofullerene ketone or the like. Specifically, by preparing an aryl fullerene dimer or a Grignard reagent, the synthesis reaction of the present disclosure can be applied for production of fullerene derivatives other than the fullerene derivative represented by the above formula (1) such as tert-butyl indanofullerene ketone or the like. The fullerene derivatives, which can be produced by the synthesis reaction of the present disclosure and are other than the fullerene derivative represented by the above formula (1), are below-described indanofullerene ketone, methyl indanofullerene ketone, methoxy indanofullerene ketone, fluoroinedanofullerene ketone, [6,6]-phenyl-C₆₁-methyl butyrate, and the like.

Moreover, the synthesis reaction of the fullerene derivative is, for example, a synthesis reaction of a fullerene derivative having an indanofullerene thioketone structure (the case where Y is sulfur) among fullerene derivatives represented by the above formula (1). This reaction is a synthesis reaction of a fullerene derivative represented by the following formula (7), in which the fullerene derivative obtained by the synthesis reaction of the fullerene derivative represented by the above formula (6) is reacted with a thionation agent.

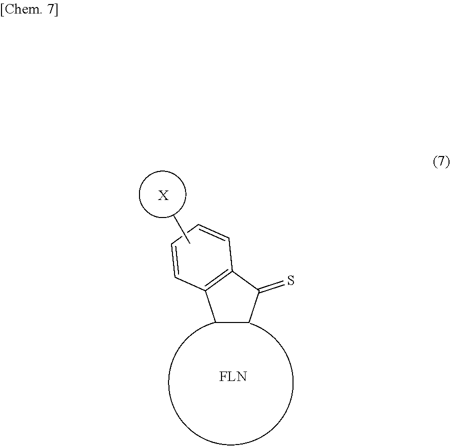

The thionation agent is not particularly limited. Examples of the thionation agent include 2,4-bis(4-methoxyphenyl)-1, 3-dithia-2, 4-diphosphetane-2, 4-disulfide (referred to as the Lawesson's reagent, hereinafter), phosphorus pentasulfide, and the like. Among the above-listed examples, the Lawesson's reagent is preferable in view of efficient transformation of a carbonyl group into a thiocarbonyl group.

The synthesis reaction of the fullerene derivative represented by the above formula (7) will be specifically described. FIG. 39 is a diagram illustrating a synthetic route of tertiary indanofullerene thioketone (tBu-FIDS).

As illustrated in FIG. 39, a mixture of tBu-FIDO (A) (210 mg, 0.238 mmol) and the Lawesson's reagent (1,350 mg, 3.341 mmol) in 50 mL of dehydrated toluene was stirred in an N₂ atmosphere at 120° C. for 24 hours. After cooling the resultant reaction solution to room temperature, 300 mL of methanol was poured into the reaction solution to precipitate a crude product. Then, the crude product obtained by filtration was subjected to silica gel column chromatography using CS₂ as an eluent to thereby obtain tBu-FIDS (B) (80 mg, 37%) as a red-brown solid.

The obtained tBu-FIDS was determined from the

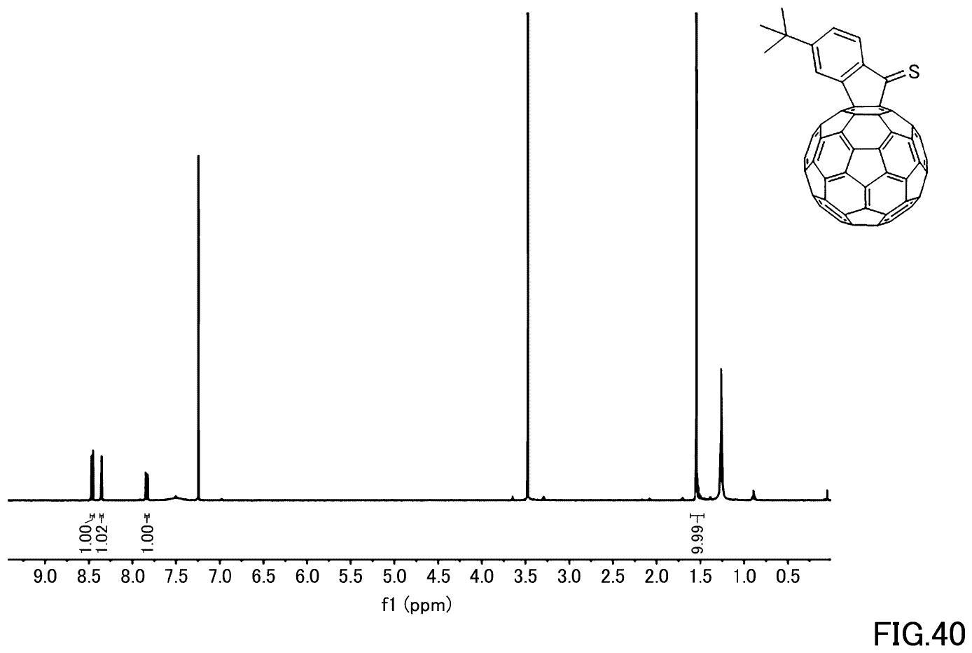

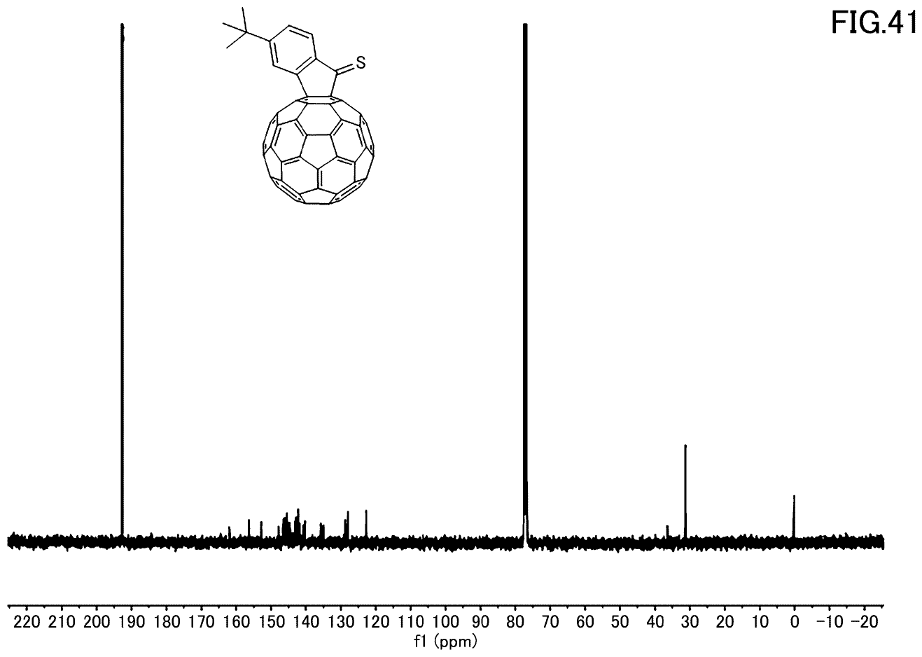

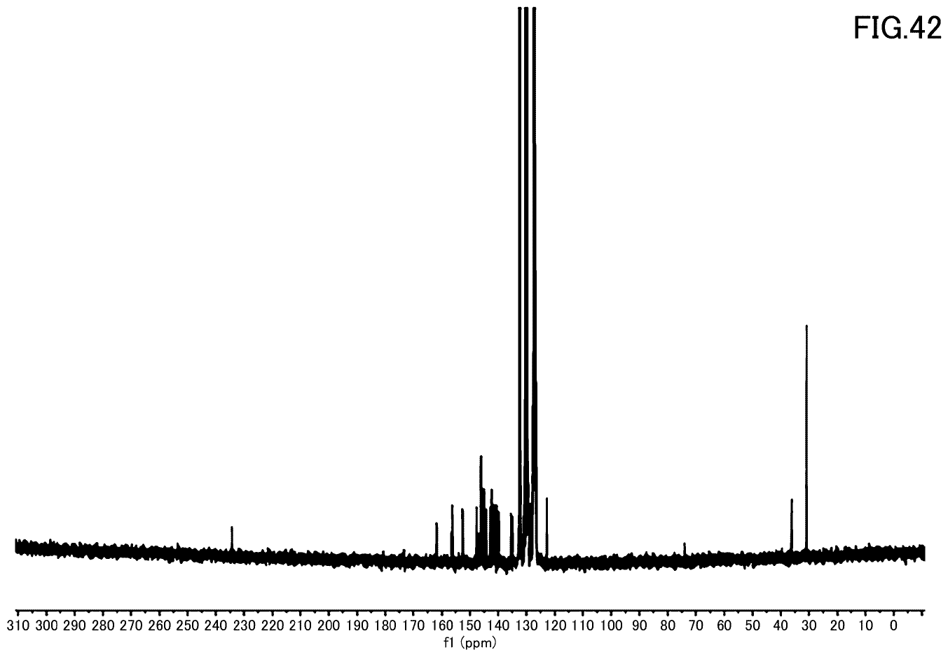

1H NMR chart (see FIG. 40) and the 13C NMR chart (see FIGS. 41 and 42). The analysis results of NMR are presented below.

1H NMR (600 MHz, CS₂/CDCl₃).

13C NMR (125 MHz, CS₂/CDCl₃, 2C unless otherwise specified).

According to the synthesis reaction of the fullerene derivative represented by the above formula (7) of the present disclosure, as described above, the fullerene derivative obtained by the synthesis reaction of the fullerene derivative represented by the above formula (6) is reacted with the thionation agent so that the fullerene derivative represented by the above formula (7), such as tert-butyl indanofullerene ketone or the like, can be synthesized at a high yield.

Moreover, according to the production method of the fullerene derivative of the present disclosure, the fullerene derivative is produced using the synthesis reaction of the fullerene derivative represented by the above formula (7) so that a vapor-deposited film having an amorphous structure can be formed using the obtained fullerene derivative.

### <Electrical Characteristics of Fullerene Derivative>

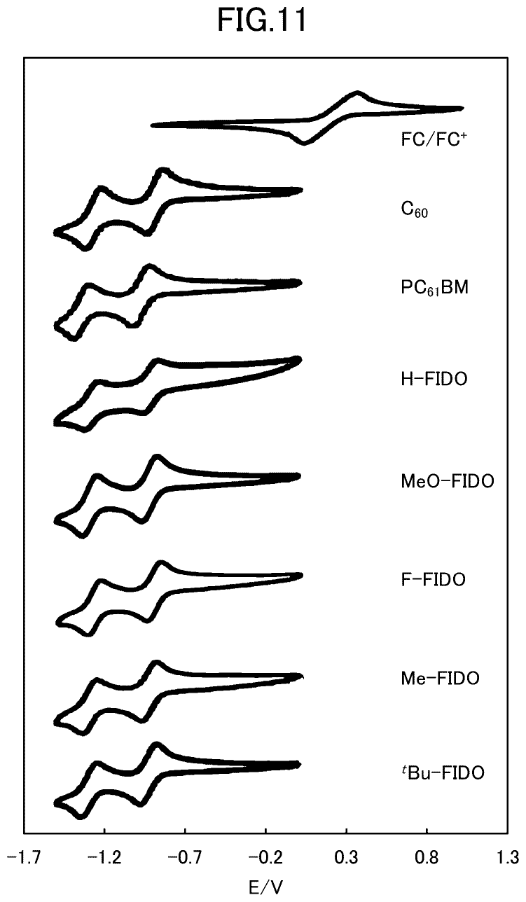

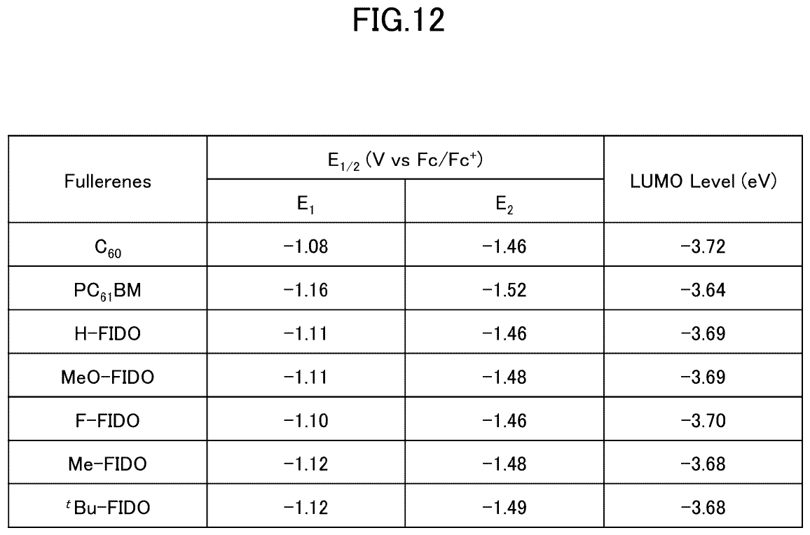

FIG. 11 is a graph illustrating reduction potential of the fullerene and the fullerene derivatives. FIG. 12 is a table illustrating the reduction potential (E_{1/2}) and electron affinity (LUMO Level (eV)) of the fullerene derivatives.

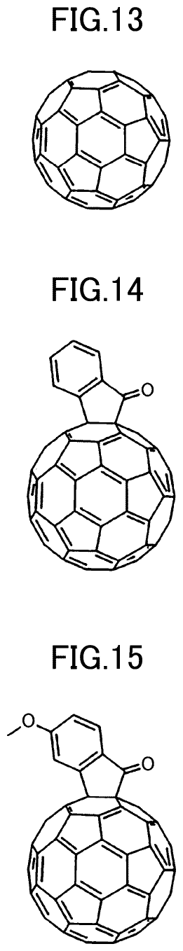

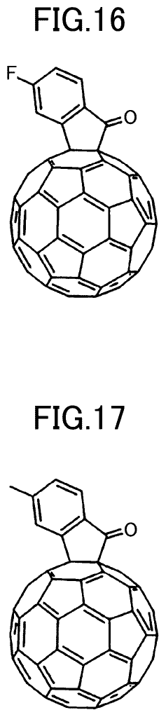

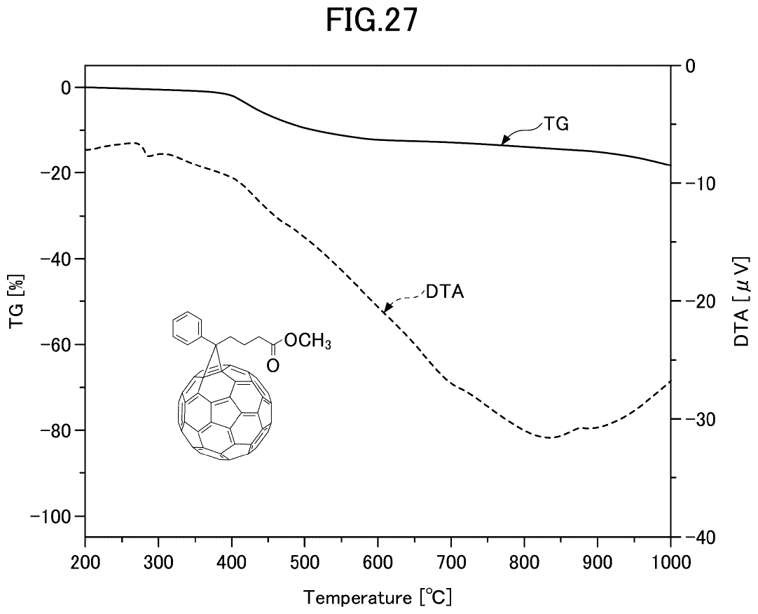

In FIGS. 11 and 12, Fc/Fc+ is a ferrocene/ferrocenium ion standard electrode, C₆₀ is a fullerene (see FIG. 13), PC₆₁BM is [6,6]-phenyl-C₆₁-methyl butyrate (see FIG. 27), H-FIDO is indanofullerene ketone (see FIG. 14), MeO-FIDO is methoxy indanofullerene ketone (see FIG. 15), F-FIDO is fluoroindanofullerene ketone (see FIG. 16), Me-FIDO is methyl indanofullerene ketone (see FIG. 17), and tBu-FIDO is tert-butyl indanofullerene ketone (see FIG. 1).

FIGS. 11 and 12 demonstrate that tBu-FIDO has the electron affinity of −3.68 and exhibits electrical characteristics comparable to the fullerene and the fullerene derivatives other than tBu-FIDO (electron affinity being −3.72 to −3.64).

Using such electrical characteristics, the fullerene derivatives, such as tBu-FIDO and the like (FIGS. 1, 14 to 17, and 27), can be applied for organic semiconductor materials and devices, such as organic light emitting diodes (OLED), organic solar cells (OSC), organic photodetectors (OPD), and the like.

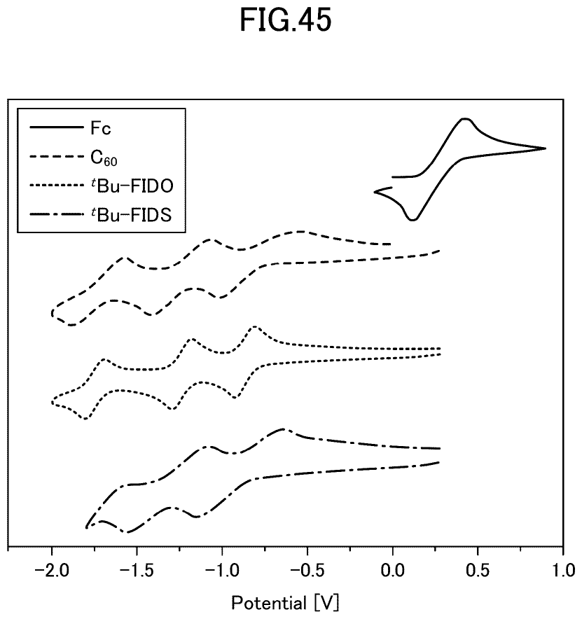

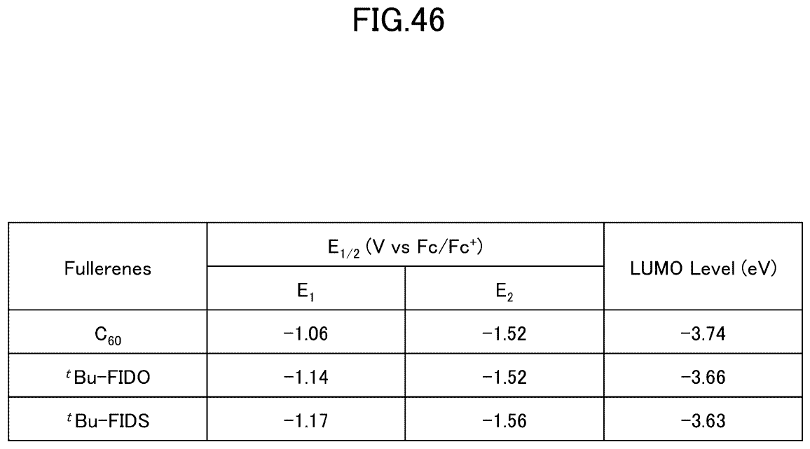

FIG. 45 is a graph illustrating reduction potential of the fullerene (C₆₀), tert-butyl indanofullerene ketone (tBu-FIDO), and tert-butyl indanofullerene thioketone (tBu-FIDS). FIG. 46 is a table illustrating reduction potential (E_{1/2}) and electron affinity (LUMO Level (eV)) of C₆₀, tBu-FIDO, and tBu-FIDS.

FIGS. 45 and 46 demonstrate that tBu-FIDI (electron affinity being −3.63) exhibit electrical characteristics comparable to Co (electron affinity being −3.74) and tBu-FIDO (electron affinity being −3.66).

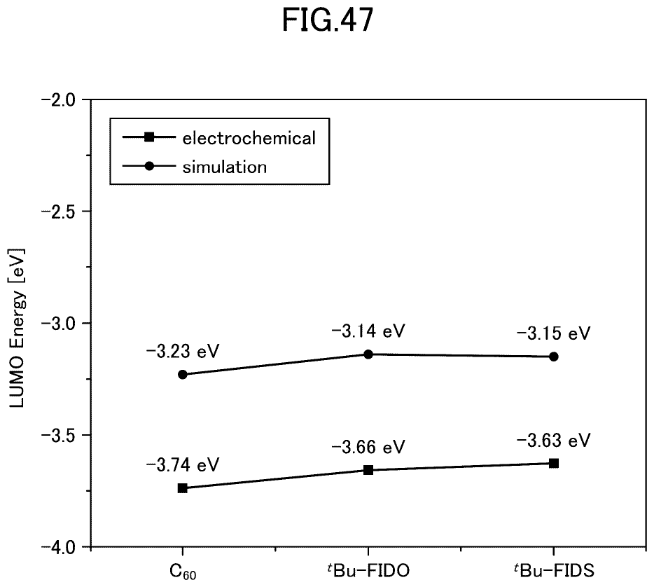

FIG. 47 is a graph illustrating simulation values and measured values of electron affinity of C₆₀, tBu-FIDO, and tBu-FIDS. The simulation values are theoretical values of the electron affinity obtained by the first-principles band calculations. FIG. 47 demonstrates that the measured values of the electron affinity of C₆₀, tBu-FIDO, and tBu-FIDS correspond to respective theoretical values.

### <Vapor-Deposited Product and Production Method of Vapor-Deposited Product>

The vapor-deposited product of the present disclosure is formed by vapor-depositing the above-described fullerene derivative of the present disclosure. The formed vapor-deposited product includes the fullerene derivative of the present disclosure. Moreover, the production method of the vapor-deposited product of the present disclosure includes vapor-depositing the fullerene derivative obtained by the above-described production method of the fullerene derivative of the present disclosure to obtain a vapor-deposited product. Note that the vapor-deposited product refers to a material formed on a deposition target, such as a substrate or the like, by vapor deposition.

As a method of vapor deposition, known film formation methods, such as physical vapor deposition (PVD) (e.g., vacuum vapor deposition, sputtering, ion plating, and the like), chemical vapor deposition (CVD) (e.g., thermal CVD, plasma CVD, optical CVD, and the like), or the like can be used. The vapor-deposited product of the present disclosure can be obtained by heating the fullerene derivative of the present disclosure at a temperature equal to or higher than the sublimation temperature.

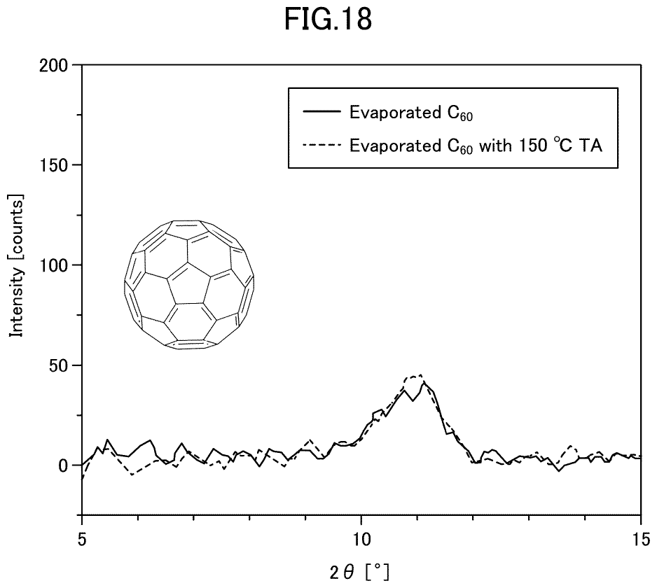

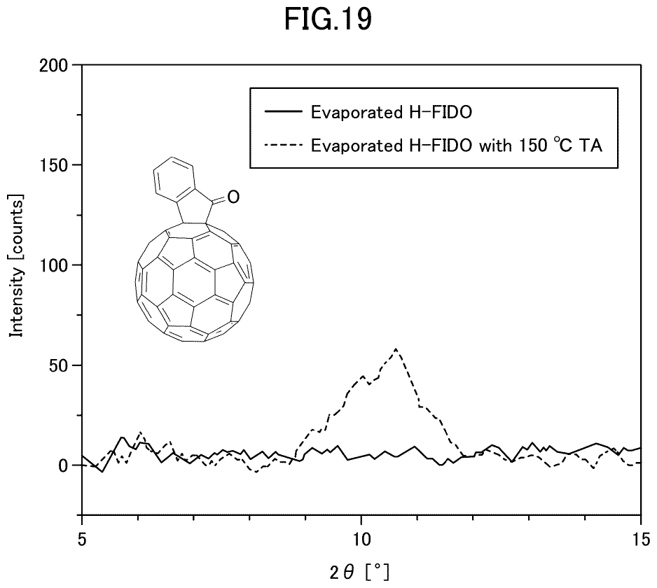

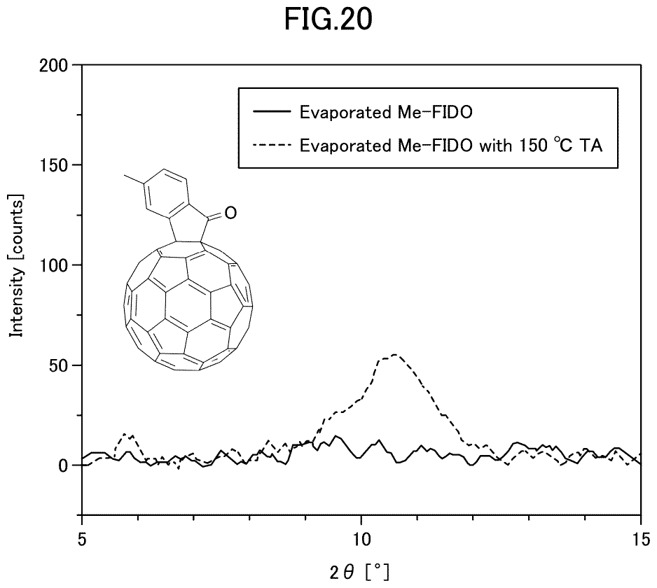

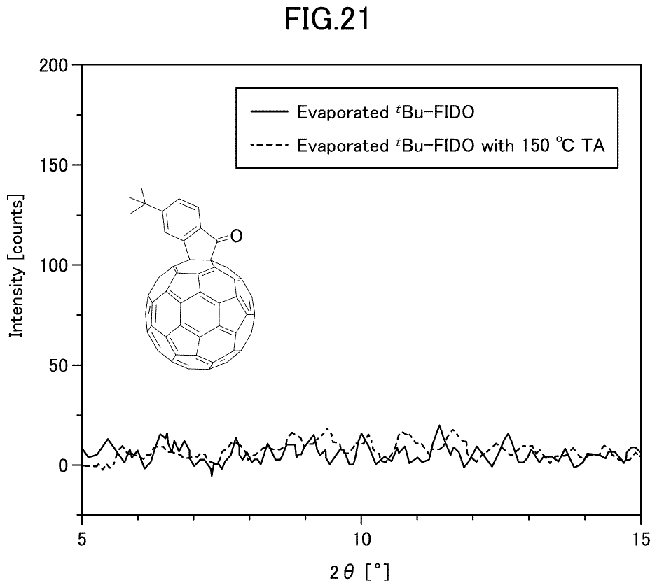

Here, the vapor-deposited product (vapor-deposited product including tBu-FIDO) of the present disclosure is compared against the vapor-deposited product including a fullerene (C₆₀) and the vapor-deposited products including the fullerene derivatives other than tBu-FIDO, such as H-FIDO, Me-FIDO, and the like based on the results of X-ray diffraction. The vapor-deposited product of Co before annealing exhibits crystallinity (see FIG. 18), and both the vapor-deposited product of H-FIDO and the vapor-deposited product of Me-FIDO before annealing exhibit an amorphous state (FIGS. 19 and 20). Comparing to the above, the vapor-deposited product of tBu-FIDO before annealing exhibits an amorphous state (see FIG. 21). In the present specification, the amorphous state refers to a state in which crystallinity is not exhibited.

In the case where a diffraction peak is confirmed in the X-ray diffraction graphs of FIGS. 19 to 21, the vapor-deposited product is determined as exhibiting crystallinity. In the case where a diffraction peak cannot be confirmed, the vapor-deposited product is determined as exhibiting an amorphous state.

As described above, the vapor-deposited product of the present disclosure is formed by vapor-depositing the fullerene derivative of the present disclosure so that the vapor-deposited product including the fullerene framework can be formed in an amorphous state, similarly to the fullerene derivatives, such as H-FIDO, Me-FIDO, and the like.

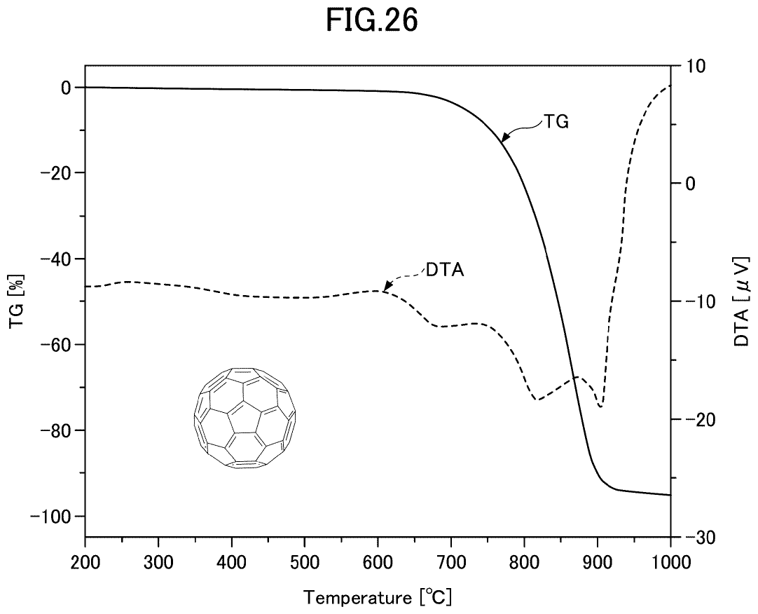

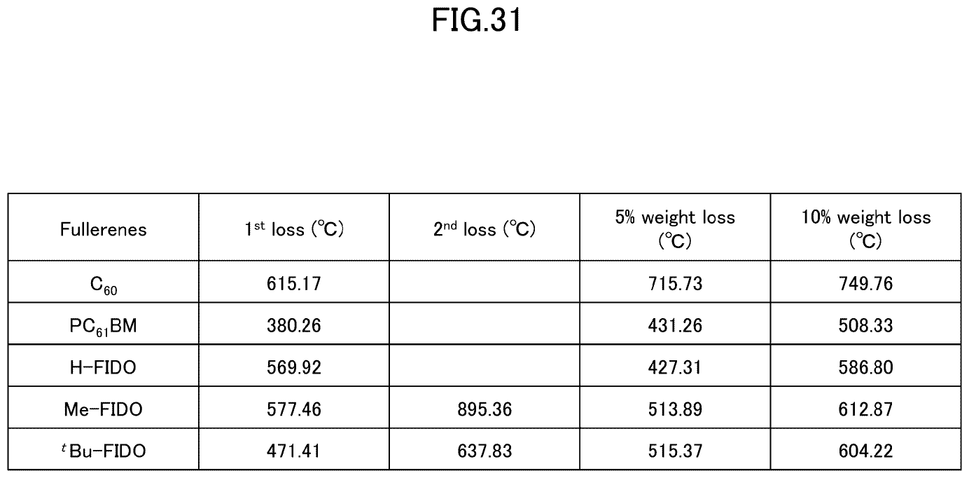

FIGS. 26 to 30 present thermal gravimetric analysis (TGA) charts of the fullerene (C₆₀) and the fullerene derivatives. FIG. 31 presents the results of TGA of the fullerene derivatives. In general, a thermal decomposition temperature of a sample is measured by thermal gravimetric analysis. In the case where a sample is sublimable, a temperature measured as a thermal decomposition onset (1st loss) temperature is the sublimation temperature of the sample.

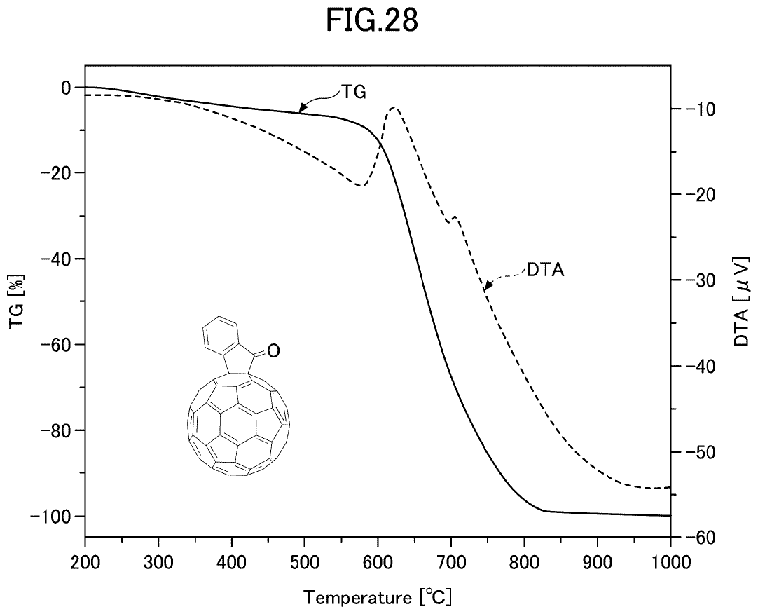

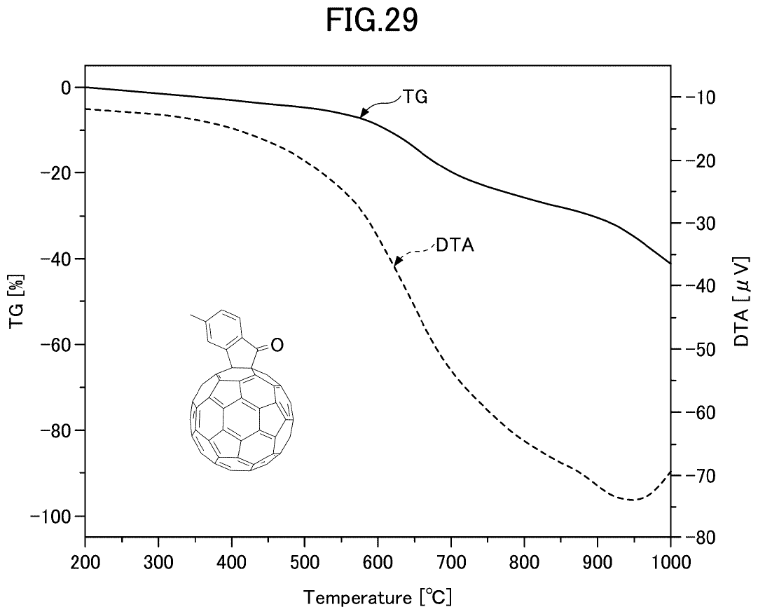

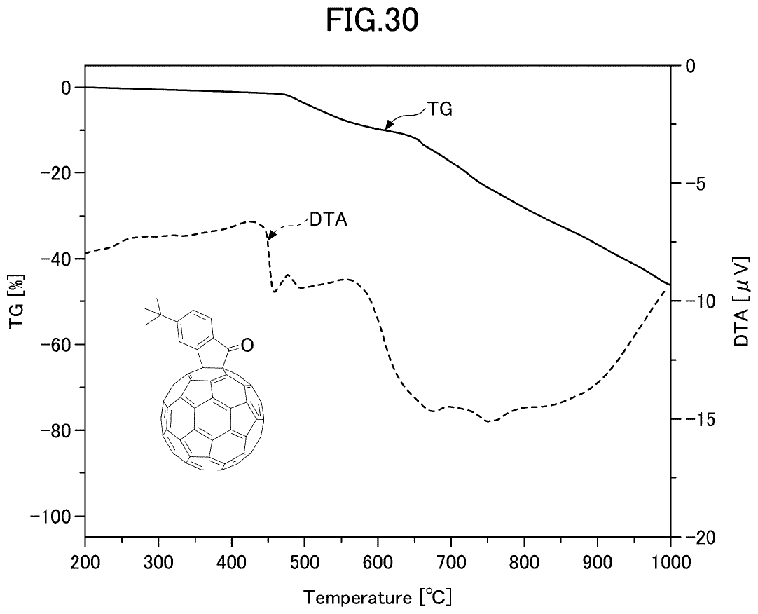

Comparing the sublimation temperatures, C₆₀ has a sublimation temperature of approximately 615° C. (see FIG. 26), [6,6]-phenyl-C₆₁-methyl butyrate (PC (BM) has a sublimation temperature of approximately 380° C. (see FIG. 27), indanofullerene ketone (H-FIDO) has a sublimation temperature of approximately 577° C. (see FIG. 28), methyl indanofullerene ketone (Me-FIDO) has a sublimation temperature of approximately 570° C. (see FIG. 29), and tert-butyl indanofullerene ketone (tBu-FIDO) has a sublimation temperature of approximately 470° C. (see FIG. 30). The above TGA demonstrates that the fullerene derivative (tBu-FIDO) of the present disclosure has a lower sublimation temperature than the fullerene (C₆₀), similarly to other fullerene derivatives.

As described above, the vapor-deposited product of the present disclosure is formed by vapor-depositing the fullerene derivative of the present disclosure so that, as the vapor-deposited product including a fullerene framework, the vapor-deposition temperature for the fullerene derivative can be made lower than the existing fullerene or fullerene derivatives, similarly to the fullerene derivatives, such as H-FIDO, Me-FIDO, and the like. Therefore, by using the vapor-deposited product of the present disclosure, a reduction in production cost of elements or devices using the fullerene derivative can be expected.

The vapor-deposited product of the present disclosure can constitute a film having an amorphous structure. The film having the amorphous structure includes the fullerene derivative of the present disclosure. The film having the amorphous structure can be formed by the above-described known film formation method.

The vapor-deposited product of the present disclosure does not exhibit crystallinity in an annealing test. In the present specification, “not exhibit crystallinity” refers to the vapor-deposited product being in an amorphous state.

Moreover, the annealing test is, as described in Experiment Examples below, a test in which a sample is subjected to annealing to evaluate crystallinity. Note that the annealing is, for example, a heat treatment performed in a nitrogen atmosphere at 150° C. for 20 minutes. Moreover, the evaluation of crystallinity is performed, for example, by grazing incidence X-ray diffraction as presented in FIGS. 18 to 21.

When a film of the vapor-deposited product (vapor-deposited film) is annealed at 150° C., the state of the vapor-deposited film of the present disclosure (tBu-FIDO) is compared against the vapor-deposited film including the fullerene (C₆₁) and the vapor-deposited films including the fullerene derivatives other than tBu-FIDO (H-FIDO, Me-FIDO) based on the results of X-ray diffraction. The vapor-deposited films of FLN, H-FIDO, and Me-FIDO (after annealing) exhibit crystallinity (see FIGS. 18 to 20). Conversely, the vapor-deposited film of tBu-FIDO (after annealing) exhibits an amorphous state (see FIG. 21). Specifically, the X-ray diffraction results of FIGS. 18 to 21 demonstrate that the vapor-deposited film of the fullerene derivative (tBu-FIDO) of the present disclosure maintains an amorphous state even after annealing.

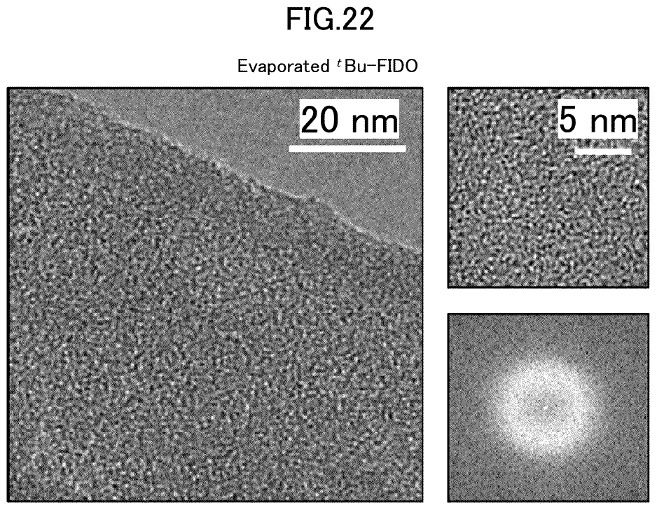

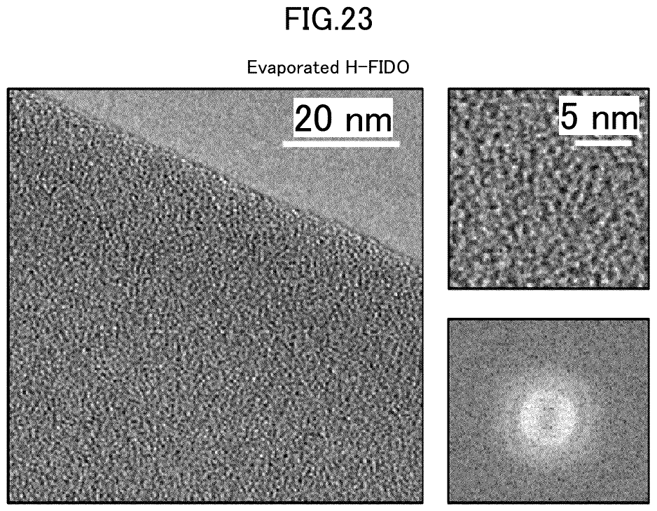

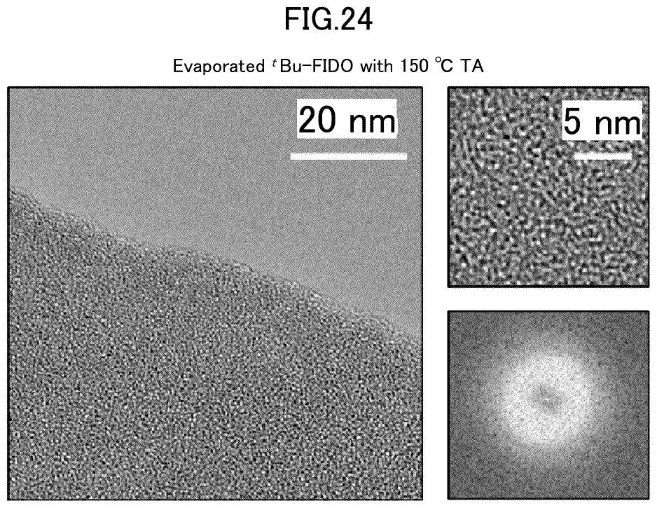

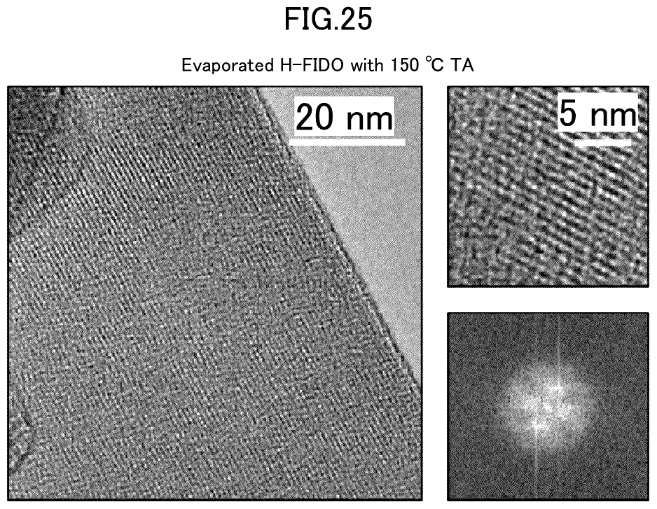

When the transmission electron microscopic (TEM) images of FIGS. 22 to 25 are compared, the vapor-deposited film of H-FIDO was in an amorphous state before annealing (see FIG. 23), but was crystallized after annealing at 150° C. (see FIG. 25). Conversely, the vapor-deposited film of tBu-FIDO was in an amorphous state before annealing (see FIG. 22), and the amorphous state was maintained even after annealing at 150° C. (see FIG. 24). Specifically, the TEM images of FIGS. 22 to 25 also demonstrate that the vapor-deposited film of the fullerene derivative (tBu-FIDO) of the present disclosure maintains the amorphous state even after annealing. Moreover, the X-ray diffraction images of FIGS. 19 and 21 are also as described above. In the case where the pattern of the image is regular in the TEM images of FIGS. 22 to 25, the vapor-deposited product is determined as exhibiting crystallinity. In the case where the pattern of the image is irregular, the vapor-deposited product is determined as exhibiting an amorphous state.

As described above, the vapor-deposited product of the present disclosure constitutes a film having an amorphous state, thus a film having an amorphous state can be formed with the fullerene derivative. Therefore, the vapor-deposited product of the present disclosure can form a highly homogeneous film of the fullerene derivative.

Moreover, the vapor-deposited product of the present disclosure does not exhibit crystallinity in the annealing test as described above so that the vapor-deposited product in the amorphous state before annealing can maintain the amorphous state also after annealing. Therefore, the film of the present disclosure can constitute a film formed for an organic element or the like using the fullerene derivative, and can be expected to be applied to semiconductor materials, devices, or the like.

Moreover, in the production method of the vapor-deposited product of the present disclosure, the fullerene derivative obtained by the production method of the fullerene derivative of the present disclosure is vapor-deposited to obtain a vapor-deposited product so that the obtained vapor-deposited product has a lower vapor-deposition temperature than the existing fullerene or fullerene derivatives. Therefore, according to the production method of the vapor-deposited product of the present disclosure, a reduction in production cost of elements or devices using the obtained fullerene derivative can be expected.

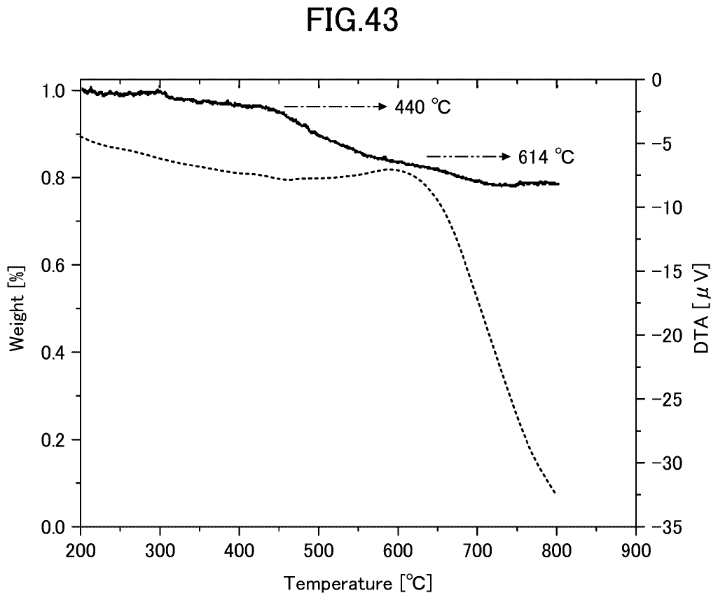

FIG. 43 presents a differential thermal analysis (DTA) chart of tBu-FIDS. In FIG. 43, an endothermic peak is not observed below 614° C. in DTA. Moreover, it can be observed that the weight of tBu-FIDS rapidly decreases at 440° C. The rapid weight loss indicates that sublimation or structural decomposition of tBu-FIDS starts at 440° C. In addition, it can be observed that tBu-FIDS starts to decompose at 614° C.

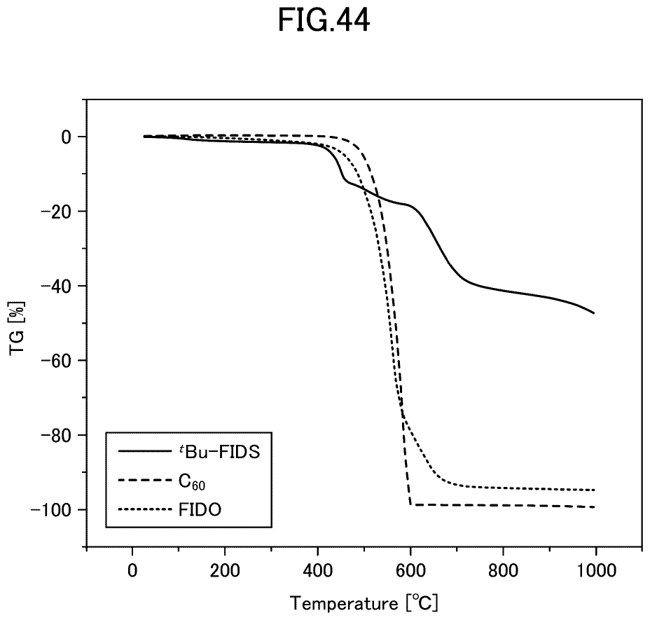

FIG. 44 presents the results of It thermogravimetry (TG) of tBu-FIDS, Coo, and tBu-FIDO. is understood from FIG. 44 that the sublimation temperature (420° C.) of tBu-FIDS is lower than the sublimation temperature (457° C.) of H-FIDO or the sublimation temperature (495° C.) of C₆₀. Moreover, the tBu-FIDS starts second weight loss at 460° C., which indicates the decomposition of the structure. Further, it can be understood that a temperature range of the weight loss of tBu-FIDS by sublimation is narrow compared to C₆₀ and tBu-FIDO.

As described above, the vapor-deposited product of the present disclosure can set a vapor-deposition temperature lower than a vapor-deposition temperature for the fullerene or tBu-FIDO even in the case where tBu-FIDS is vapor-deposited. Thus, a reduction in production cost of elements or devices using tBu-FIDS can be expected.

Moreover, in the production method of the vapor-deposited product, the vapor-deposition temperature of tBu-FIDS can be made lower than the vapor-deposition temperature for fullerene or tBu-FIDO even in the case where tBu-FIDS is used so that a reduction in production cost of elements or devices using tBu-FIDS can be expected.

### EXAMPLES

The present disclosure will be further described through experiment examples hereinafter. The tests and evaluations were performed in the following manner.

### Experiment Example 1

As a fullerene derivative, tert-butyl indanofullerene ketone (tBu-FIDO) was used (see FIG. 1 and FIG. 30). In the present example, tert-butyl indanofullerene ketone may be referred to as tert-butyl fullerene ketone.

### Experiment Example 2

A fullerene (referred to as FLN or C₆₀ hereinafter) was used instead of a fullerene derivative (see FIG. 13 and FIG. 26).

### Experiment Example 3

As a fullerene derivative, indanofullerene ketone (H-FIDO) was used (see FIG. 14 and FIG. 28). In the present example, the indanofullerene ketone may be referred to as fullerene ketone.

### Experiment Example 4

As a fullerene derivative, methyl indanofullerene ketone (Me-FIDO) was used (see FIG. 17 and FIG. 29).

### Experiment Example 5

As a fullerene derivative, fluoroindanofullerene ketone (F-FIDO) was used (see FIG. 16).

### Experiment Example 6

As a fullerene derivative, methoxy indanofullerene ketone (MeO-FIDO) was used (see FIG. 15).

### Experiment Example 7

As a fullerene derivative, [6,6]-phenyl-C₆₁-methyl butyrate (PC (BM) was used (see FIG. 27).

### <Vapor-Deposited Product>

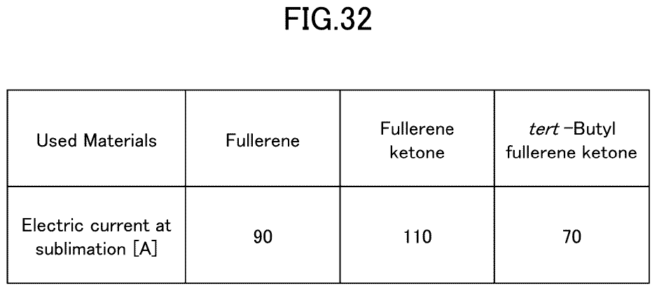

Using a vacuum deposition device (VTS-350M, manufactured by ULVAC, Inc.), an electric current value of a heater for heating a crucible charged with a material to be vapor-deposited was gradually increased in high vacuum (4.76×10-3 Pa) to sublimate the material, to thereby produce a vapor-deposited film of approximately 50 nm. The electric current value applied at the time when the vapor-deposited film started to be formed is presented in FIG. 32.

### <Annealing>

A sample that was formed as a film was heated in a nitrogen atmosphere at 150° C. for 20 minutes. Specifically, the film of the sample formed on a quartz substrate was set on a hotplate heated at 150° C. in a glove box purged with nitrogen, the sample was covered with a petri dish, and a heat treatment was performed for 20 minutes. In the case where annealing was performed in a solvent vapor, the sample and a small bottle charged with chlorobenzene serving as a solvent were set in the petri dish, and a heat treatment was similarly performed at 150° C. for 20 minutes.

### <X-Ray Diffraction>

A sample formed as a film having a film thickness of approximately 30 nm on a quartz substrate was set on a thin film X-ray diffractometer (ATX-G, manufactured by RIGAKU Corporation), and measured by grazing incidence X-ray diffraction.

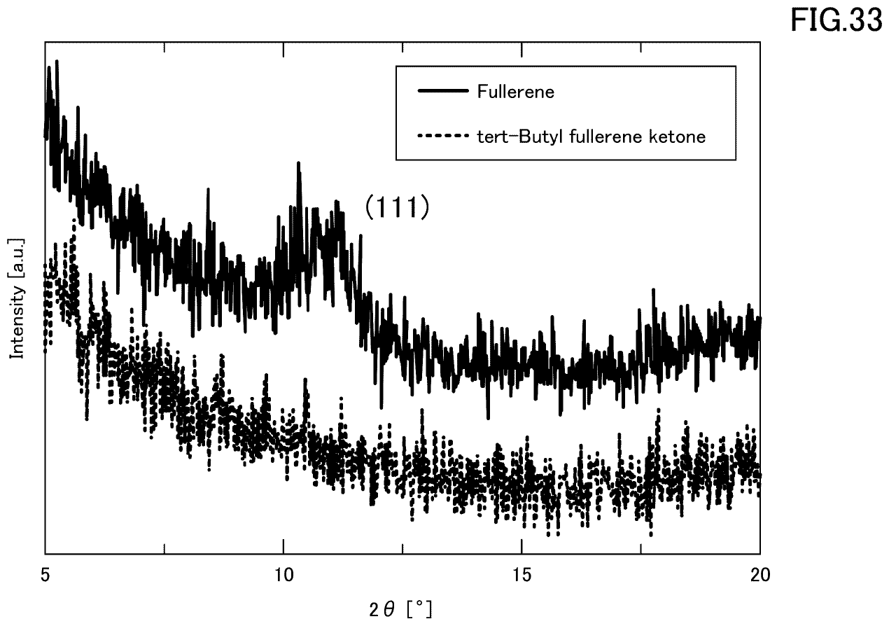

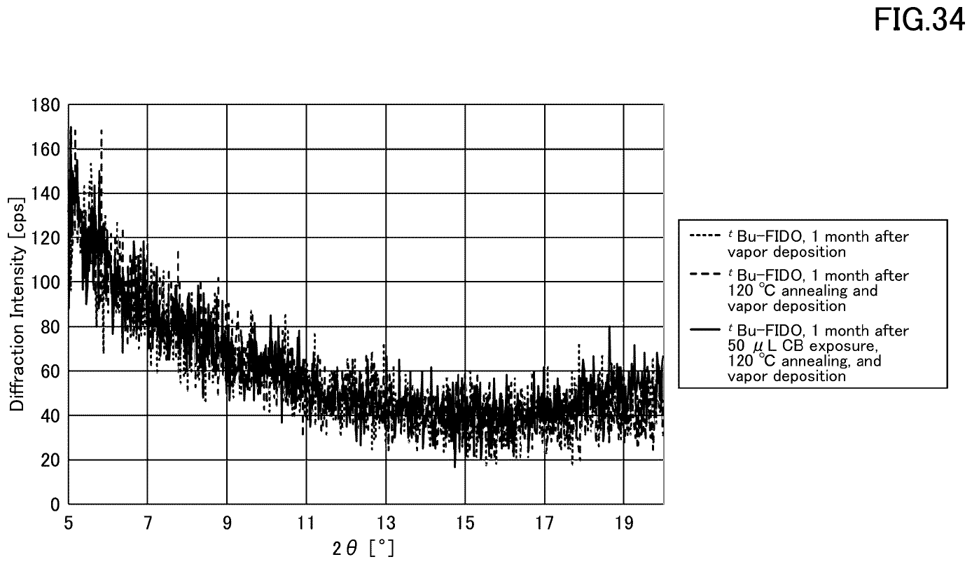

The results of X-ray diffraction demonstrate that the vapor-deposited product of Experiment Example 1 (tBu-FIDO) before annealing exhibited an amorphous state, and the vapor-deposited product also exhibited an amorphous state one month after the vapor deposition (see FIGS. 21, 33, and 34). In addition, the vapor-deposited product after annealing exhibited an amorphous phase, and also exhibited an amorphous state one month after the annealing (see FIGS. 21 and 34). Further, the vapor-deposited product after annealing exhibited an amorphous state in a solvent vapor, and also exhibited an amorphous state one month after the annealing (see FIG. 34).

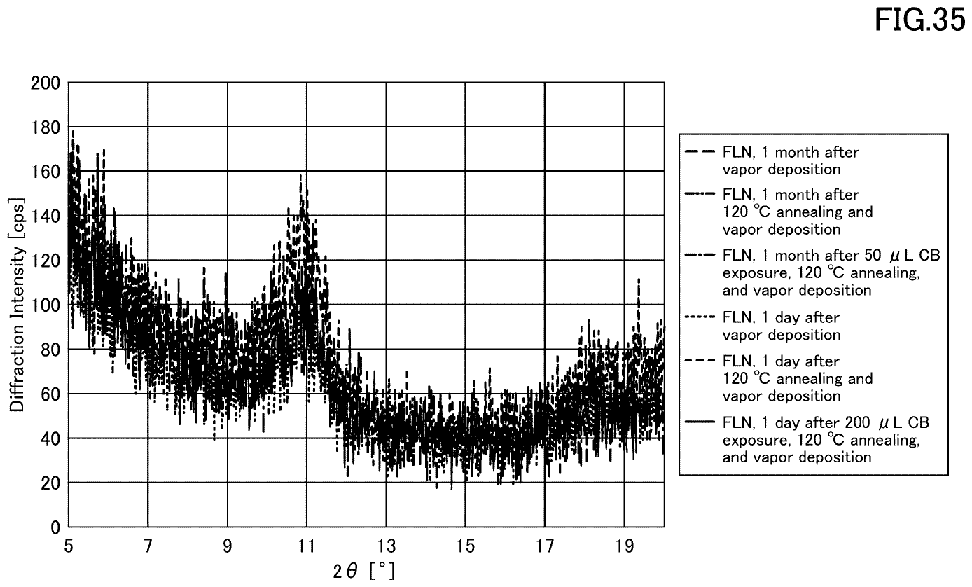

Conversely, in Experiment Example 2 (FLN), the vapor-deposited product before annealing exhibited crystallinity, and the vapor-deposited product also exhibited crystallinity one month after the vapor deposition (see FIGS. 21, 33, and 35). In addition, the vapor-deposited product after annealing exhibited crystallinity, and also exhibited crystallinity one month after the annealing (see FIGS. 21 and 34). Further, the vapor-deposited product after annealing exhibited crystallinity in a solvent vapor, and also exhibited crystallinity one month after the annealing (see FIG. 35).

Moreover, in both Experiment Example 3 (H-FIDO) and Experiment Example 4 (Me-FIDO), the vapor-deposited product before annealing exhibited an amorphous state, but the vapor-deposited product after annealing exhibited crystallinity (see FIGS. 19 and 20).

It was found from the above results that the fullerene derivative of Experiment Example 1 (tBu-FIDO) enabled the vapor-deposited product of the fullerene derivative to maintain the amorphous state, which was the state immediately after vapor deposition, after annealing, and the amorphous state could be maintained for a long period before and after the annealing regardless of the presence of a solvent vapor. Accordingly, film formation for an organic element or the like using the fullerene derivative of Experiment Example 1 (tBu-FIDO) can be easily performed, and therefore application of the fullerene derivative of Experiment Example 1 (tBu-FIDO) for semiconductor materials, devices, or the like can be expected.

### <Transmission Electron Microscope (TEM) Image>

A sample formed as a film on a quartz substrate was set in a transmission electron microscope, and TEM measurement was performed.

The results of the TEM images demonstrate that, in Experiment Example 1 (tBu-FIDO), the vapor-deposited product before annealing exhibited an amorphous state (see FIG. 22), and the vapor-deposited product after annealing also exhibited an amorphous state (see FIG. 24). Conversely, in Experiment Example 3 (H-FIDO), the vapor-deposited product before annealing exhibited an amorphous state (FIG. 23), but the vapor-deposited product after annealing exhibited crystallinity (see FIG. 25).

It was also found from the above results of the TEM images that the fullerene derivative of Experiment Example 1 (tBu-FIDO) enabled the vapor-deposited product of the fullerene derivative to maintain the amorphous state, which was the state immediately after vapor deposition, after annealing, and film formation of an organic element or the like using the fullerene derivative could be easily performed.

### <Thermal Gravimetric Analysis (TGA)>

A sample approximately (approximately 5 mg) was set in a thermal gravimetric analyzer (DTG-60AH, Shimadzu Corporation). The temperature was increased from room temperature to 100° C. at the rate of 10° C./min under a nitrogen stream, and the temperature was maintained at 100° C. for 10 minutes. Thereafter, the temperature was increased from 100° C. to 1, 000° C. at the rate of 10° C./min to thereby measure a change in weight. The results at 100° C. or higher are presented in FIGS. 26 to 30.

According to the results of TGA, the sublimation temperature of the vapor-deposited product was 471.41° C. (see FIGS. 30 and 31) in Experiment Example 1 (tBu-FIDO). Compared with the above, the sublimation temperature was 615.17° C. in Experiment Example 2 (C₆₀), 569.92° C. in Experiment Example 3 (H-FIDO), 577.46° C. Experiment Example 4 (Me-FIDO), and 380.26° C. in Experiment Example 7 (PC₆₁BM) (see FIGS. 26 to 29 and 31).

It was found from the above results that the fullerene derivative of Experiment Example 1 (tBu-FIDO) had a lower sublimation temperature than the fullerene (C₆₀), similarly to the existing fullerene derivatives. Therefore, the fullerene derivative of Experiment Example 1 (tBu-FIDO) can use a lower vapor-deposition temperature than the fullerene (C₆₀), similarly to the existing fullerene derivatives so that a reduction in production cost of elements or devices using the fullerene derivative can be expected.

### <Thermogravimetry (Vacuum TG)>

A sample (approximately 5 mg) was set in a vacuum thermogravimetry analyzer (TG/DTA6200, manufactured by Hitachi High-tech Analysis Corporation), and the change in weight of the sample was measured in a vacuum of 1 Pa or lower by heating the sample from room temperature to 1,000° C. at the rate of 10° C./min.

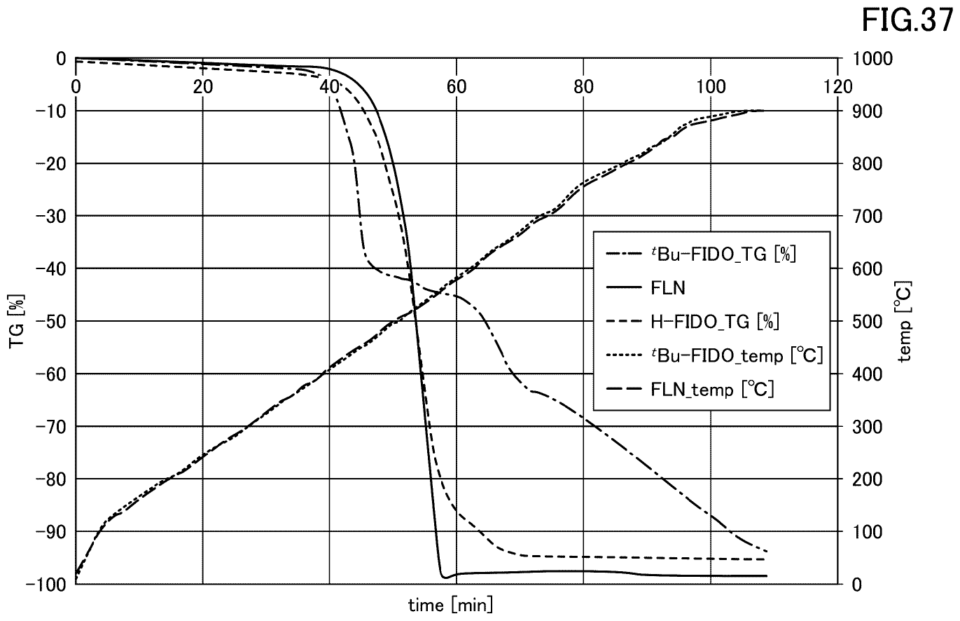

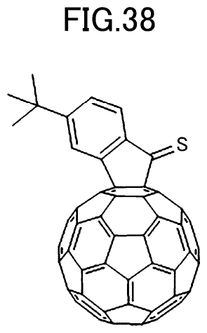

It was found from the results of the vacuum TG that the weight loss was accelerated in Experiment Example 1 (tBu-FIDO) by sublimation compared to Experiment Example 2 (C₆₀) and Experiment Example 3 (H-FIDO) (see FIG. 37).

It was found from the above results that the fullerene derivative of Experiment Example 1 (tBu-FIDO) had a shorter sublimation onset time than the existing fullerene or fullerene derivatives under the same temperature elevation condition. Therefore, use of the fullerene derivative of Experiment Example 1 (tBu-FIDO) can shorten the vapor-deposition time compared to the existing fullerene or fullerene derivatives. Thus, a reduction in production cost of elements or devices using the fullerene derivative can be expected.

### <Measurement of Electric Current at the Time of Sublimation>

When the above-described vapor-deposited product was produced, the electric current value applied to the crucible was measured, and the electric current value at the time the vapor-deposited film started to generate was determined as electric current at the time of sublimation (see FIG. 32).

The electric current at the time of sublimation was 70 A in Experiment Example 1 (tBu-FIDO). Compared to the above, the electric current at the time of sublimation was 90 A in Experiment Example 2 (C₆₀), and 110 A in Experiment Example 3 (H-FIDO) (see FIG. 32). It is expected from the above results that the sublimation temperature of Experiment Example 1 (tBu-FIDO) is lower than a sublimation temperature for the existing fullerene or fullerene derivatives.

Therefore, use of the fullerene derivative of Experiment Example 1 (tBu-FIDO) can lower the vapor-deposition temperature and reduce the vapor-deposition time compared to the existing fullerene or fullerene derivatives. Thus, a reduction in production cost of elements or devices using the fullerene derivative can be expected.

### <Ultraviolet-Visible Spectroscopy (UV-Vis)>

A sample that was formed as a film having a thickness of approximately 30 nm on a quartz substrate was set in an ultraviolet-visible spectrophotometer (V-650, manufactured by JASCO Corporation), and measurement was performed in a wavelength range of 200 nm to 900 nm.

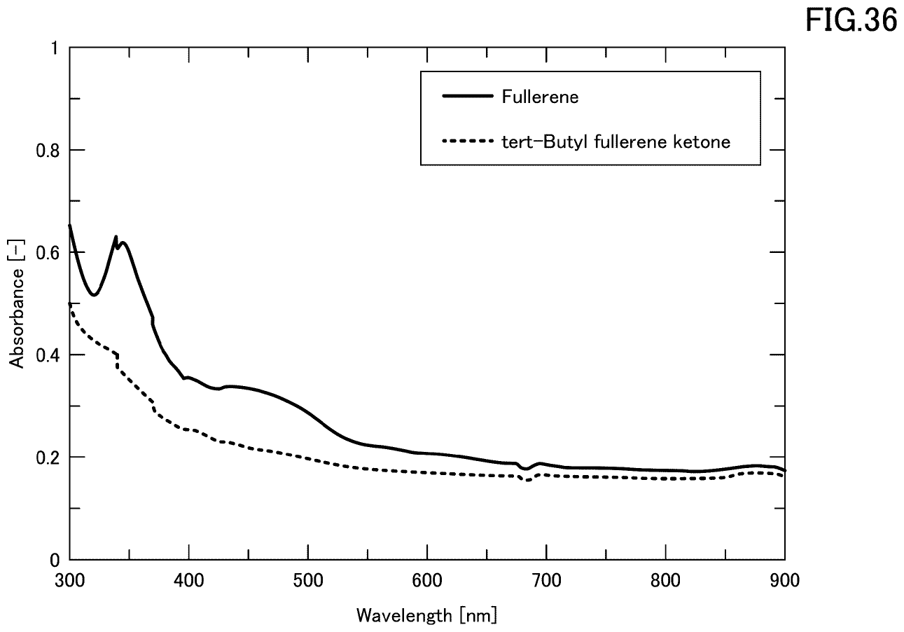

From the results of the ultraviolet-visible spectroscopy, for example, at the wavelength of 450 nm (blue), the absorbance of Experiment Example 1 (tBu-FIDO) was approximately 0.2 (see FIG. 36). Conversely, the absorbance of Experiment Example 2 (C₆₀) was approximately 0.3 (see FIG. 36).

The above results demonstrate that the fullerene derivative of Experiment Example 1 (tBu-FIDO) has a property that is less likely to absorb the blue wavelength. Therefore, the vapor-deposited product of Experiment Example 1 (tBu-FIDO) can be used as, for example, an acceptor of an image sensor.

Although the embodiments of the present invention have been described above, the present invention is not limited to the specific embodiments, and various modifications and changes can be made within the scope of the invention described in the claims.

This application claims priority to Japanese Patent Application No. 2023-048815 filed on Mar. 24, 2023 and Japanese Patent Application No. 2023-205171 filed on Dec. 5, 2023, the entire contents of which are incorporated herein by reference.

1. A fullerene derivative, represented by a formula (1): 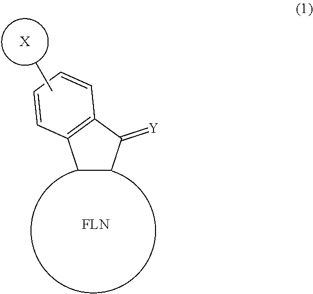
in the formula (1), FLN is a fullerene framework, X is a halogen group or an organic group, and Y is O or S.

2. The fullerene derivative according to claim 1,
wherein the fullerene derivative is represented by a formula (2): 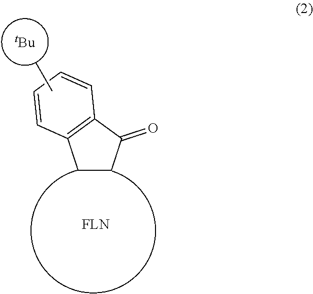
in the formula (2), tBu is a tert-butyl group.

3. The fullerene derivative according to claim 2,
wherein the fullerene derivative is represented by a formula (3):

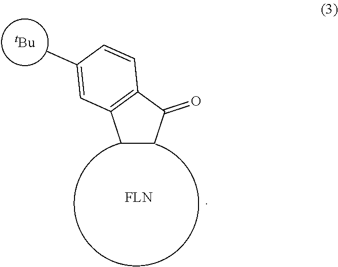

4. The fullerene derivative according to claim 1,
wherein the fullerene derivative is represented by a formula (4): 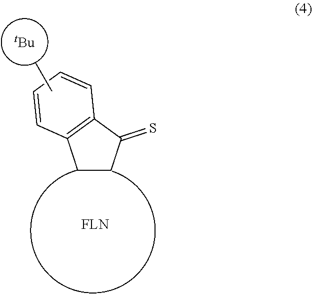
in the formula (4), tBu is a tert-butyl group.

5. The fullerene derivative according to claim 4,
wherein the fullerene derivative is represented by a formula (5):

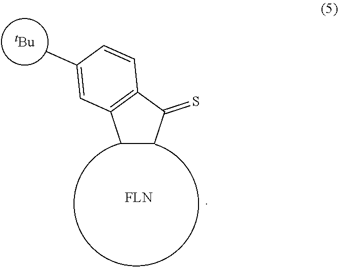

6. The fullerene derivative according to claim 1,
wherein the fullerene framework is a C₆₀ framework, a C₇₀ framework, a C₇₆ framework, or a C₇₈ framework.

7. A vapor-deposited product, obtained by a method comprising:
vapor-depositing the fullerene derivative according to claim 1.

8. The vapor-deposited product according to claim 7,
wherein the vapor-deposited product is a film having an amorphous structure.

9. The vapor-deposited product according to claim 7,
wherein the vapor-deposited product does not exhibit crystallinity in an annealing test, where the annealing test includes: performing a heat treatment on the vapor-deposited product in a nitrogen atmosphere at 150° C. for 20 minutes, followed by performing grazing incidence X-ray diffraction to evaluate crystallinity of the vapor-deposited product.

10. A synthesis reaction of a fullerene derivative represented by a formula (6), the synthesis reaction comprising:
oxidizing an aryl fullerene dimer in the presence of hydrohalic acid, 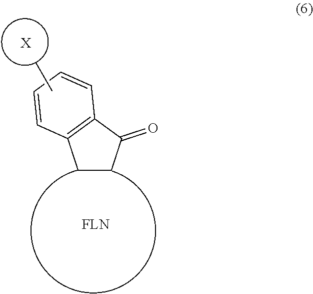
in the formula (6), FLN is a fullerene framework, and X is a hydrogen, a halogen group, or an organic group.

11. The synthesis reaction of the fullerene derivative according to claim 10,
wherein the hydrohalic acid is hydrobromic acid.

12. A synthesis reaction of a fullerene derivative represented by a formula (7), the synthesis reaction comprising:
reacting a fullerene derivative obtained by the synthesis reaction of claim 10 with a thionation agent, 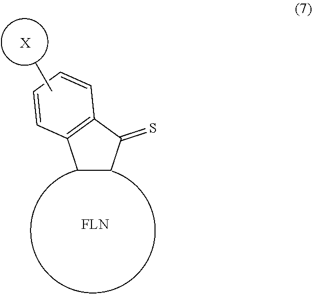
in the formula (7), FLN is a fullerene framework, and X is a halogen group or an organic group.

13. A production method of a fullerene derivative, the production method comprising:
producing a fullerene derivative using the synthesis reaction according to claim 10.

14. A production method of a vapor-deposited product, the production method comprising:
obtaining a fullerene derivative by the production method of claim 13, and vapor-depositing the fullerene derivative to obtain a vapor-deposited product.
